(19) United States
(12) Patent Application Publication
(10) Pub. No.: US 2018/0318260 A1
(43) Pub. Date: Nov. 8, 2018

(54) COMPOSITIONS AND METHODS FOR TREATING CYSTIC FIBROSIS

(71) Applicant: THE SCRIPPS RESEARCH INSTITUTE, La Jolla, CA (US)

(72) Inventors: Sandra PANKOW, Del Mar, CA (US); Casimir BAMBERGER, Del Mar, CA (US); John YATES, San Diego, CA (US)

(21) Appl. No.: 15/773,427

(22) PCT Filed: Nov. 4, 2016
(86) PCT No.: PCT/US2016/060515

§ 371 Date: May 3, 2018

Related U.S. Application Data
(60) Provisional application No. 62/250,603, filed on Nov. 4, 2015.

(51) Int. Cl. A61K 31/417; A61K 31/4184; A61K 31/7105; A61K 31/713; A61P 11/00

(52) U.S. Cl. CPC A61K 31/417; A61K 31/4184; A61P 11/00; A61K 31/713; A61K 31/7105

(57) ABSTRACT

This invention provides compositions and methods for restoring proper folding and function of Cystic Fibrosis Transmembrane Conductance Regulator mutant with in-flame-deletion of phenylalanine 508 (AF508 CFTR). The invention also provides methods for identifying novel agents capable of restoring proper folding and function of ΔP508 CFTR. The invention additionally provides methods for treating cystic fibrosis.

### CROSS-REFERENCE TO RELATED APPLICATIONS

The subject patent application claims the benefit of priority to U.S. Provisional Patent Application Number 62/250,603 (filed Nov. 4, 2015). The full disclosure of the priority application is incorporated herein by reference in its entirety and for all purposes.

### STATEMENT CONCERNING GOVERNMENT SUPPORT

This invention was made with government support under HL079442 awarded by the National Institutes of Health. The government has certain rights in the invention.

### BACKGROUND OF THE INVENTION

Cystic Fibrosis (CF), also called mucoviscidosis, is the most common lethal genetic disorder in Caucasians. This incurable disease is caused by mutation of the Cystic Fibrosis Transmembrane Conductance Regulator (CFTR) gene, which encodes a 12 transmembrane chloride channel critical for salt homeostasis of polarized epithelial tissues such as the lung, intestine, pancreas and kidney, CF patients suffer from unusually sticky mucus, which promotes chronic lung infections eventually leading to respiratory failure. The most prevalent mutation occurring in more than 70% of patients is an in-frame-deletion of phenylalanine 508 (ΔF⁵⁰⁸ CFTR). While the ΔF⁵⁰⁸ CFTR protein is fully expressed and is in principle functional as shown by rescue of ΔF⁵⁰⁸ CFTR channel activity at low temperature (23-30°C.), the deletion of F⁵⁰⁸ causes the protein to be energetically unstable, preventing correct folding and trafficking. Instead, ΔF⁵⁰⁸ CFTR is degraded by the ER associated degradation pathway (ERAD). CF is therefore characterized as a protein misfolding disease. Current therapies are directed to symptoms and the most promising drugs Lumacaftor (VX-809) and Ivacaftor (VX-770) showed only relatively minor improvement of ΔF508 CFTR function in clinical trials so far. In addition, responses of individual patients to these drugs vary significantly.

There is a strong and urgent need in the art for better means and more effective drugs for treating cystic fibrosis. The present invention addresses this and other unfulfilled needs in the art.

### SUMMARY OF THE INVENTION

In one aspect, the invention provides methods for restoring proper folding and function of Cystic Fibrosis Transmembrane Conductance Regulator (CFTR) in a cell expressing or harboring phenylalanine 508 in-flame-deletion CFTR mutant (ΔF⁵⁰⁸ CFTR). The methods entail contacting the cell harboring the mutant CFTR with an agent that specifically downregulates expression level, inhibits cellular activity, or interrupts protein-protein interaction of a CFTR interactor protein. This enables restoration of proper folding and function of CFTR in the cell. In these methods, the referenced CFTR interactor protein is selected from the group consisting of glutaminyl-peptide cyclotransferase (QPCT), polyadenylate-binding protein 1 (PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), ES ubiquitin-protein ligase TRIM21 (TRIM21), protein-tyrosine phosphatase-like A domain-containing protein 1 (PTPLAD1), protein-glutamine gamma-glutamyltransferase 2 (TGM2), and RelA-associated inhibitor (PPP1R13).

In some methods, the employed agent specifically inhibits the cellular activity of the CFTR interactor protein. For example, the CFTR interactor protein can be an enzyme, and the agent specifically inhibits the enzymatic activity of the CFTR interactor protein. In some of these methods, the CFTR interactor protein is glutaminyl peptide cyclotransferase (QPCT), and the agent is a small molecule inhibitor of QPCT. The small molecule inhibitor of QPCT used in these methods can be, e.g., N-ω-acetylhistamine (systematic name: N-[2-(1H-Imidazol-4-yl)ethyl]acetamide, CAS: 673-49-4), 1-benzylimidazole (systematic name: 1-Benzyl-1H-imidazole; CAS: 4238-71-5), 3-(3,4-dimethoxyphenyl)-1-(3-imidazol-1-ylpropyl)thiourea, 1-(3,4-Dimethoxyphenyl)-N-(3-(5-methyl-1H-imidazol-1-yl)propyl)cyclopropanecarbothioamide, a derivative or analog compound thereof, or a pharmaceutically acceptable salt thereof. In some methods, the derivative or analog compound has one or more mono- or multi-valent groups substituted with a different mono- or multi-valent group independently selected from the group consisting of: H; halogen; straight, cyclic or branched chain alkyl; straight, cyclic or branched chain alkenyl; straight, cyclic or branched chain alkynyl; -alkenyl or -alkynyl; CN; CF₃; aryl and substituted aryl groups in which any or all H groups of the aryl ring is substituted with a different group; heterocyclic and substituted heterocyclic groups in which any or all groups of the aryl ring is substituted with a different group; carboxyl; carbonyl, alkoxyl; alkyloxyalkanes; alkoxycarbonyl; aryloxyl, heterocyclyloxyl; hydroxyl; amine; amide; amino; quaternary amino; nitro; sulfonyl; alkylamine; silyl, siloxyl; saturated C—C bonds; unsaturated C—C bonds; ester, ether, amino; amide, urethane, carbonyl, acetyl and ketyl groups; hetero atoms N, S and O; polymer groups; and amino acids. In some embodiments, the derivative or analog compound has one or more hydrogens substituted with a lower alkyl group.

In some methods of the invention, the employed agent specifically downregulates expression level of the CFTR interactor protein. Some of these methods employ an inhibitory polynucleotide that suppresses expression of the CFTR interactor protein. Examples of inhibitory polynucleotides that can be used include short hairpin RNA (shRNA), short interfering RNAs (siRNA), microRNAs (miRNA), and an anti-sense nucleic acid. Some methods of the invention are directed to pulmonary epithelial cells that express or harbor a ΔF⁵⁰⁸ CFTR mutant. In some of these methods, the pulmonary epithelial cells are present in a subject, e.g., a subject afflicted with cystic fibrosis.

In a related aspect, the invention provides methods for treating or ameliorating the symptoms of cystic fibrosis in a subject. The methods involve administering to the subject a pharmaceutical composition that contains a therapeutic effective amount of an agent that specifically dog regulates expression level or inhibits cellular activity of a CFTR interactor protein. This enables treatment or amelioration of symptoms of cystic fibrosis in the subject. In these methods, the CFTR interactor protein is selected from the group consisting of glutaminyl-peptide cyclotransferase (QPCT), polyadenylate-binding protein 1 (PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), E3 ubiquitin-protein ligase TRIM21 (TRIM21), protein-tyrosine phosphatase like A domain-containing protein 1 (PTPLAD1), protein-glutamine gamma-glutamyltransferase 2 (TGM2), and RelA-associated inhibitor (PPPIR13). In various embodiments, the subject to be treated with the methods expresses or harbors a Cystic Fibrosis Transmembrane Conductance Regulator (CFTR) mutant with phenylalanine 508 in-flame-deletion (ΔF⁵⁰⁸ CFTR).

In some of these therapeutic methods, the employed agent specifically inhibits the cellular activity of the CFTR interactor protein. For example, the CFTR interactor protein can be an enzyme, and the agent specifically inhibits the enzymatic activity of the CFTR interactor protein. In some of these methods, the CFTR interactor protein is glutaminyl-peptide cyclotransferase (QPCT), and the agent is a small molecule inhibitor of QPCT. Examples of small molecule inhibitors of QPCT that may be used include, e.g., N-ω-acetylhistamine (systematic name: N-[2-(1H-Imidazol-4-yl)ethyl]acetamide, CAS: 673-49-4), 1-benzylimidazole (systematic name: 1-Benzyl-1H-imidazole; CAS: 4238-71-5), 3-(3,4-dimethoxyphenyl)-1 -(3-imidazol-1-ylpropyl)thiourea, 1-(3,4-Dimethoxyphenyl)-N-(3-(5-methyl-1H-imidazol-1-yl)propyl)cyclopropanecarbothioamide, a derivative or analog compound thereof, or a pharmaceutically acceptable salt thereof. In some other therapeutic methods, the employed agent specifically downregulates the expression level of the CFTR interactor protein. For example, the employed agent can be an inhibitory polynucleotide that suppresses expression of the CFTR interactor protein. Examples of suitable inhibitory polynucleotides include short hairpin RNA (shRNA), short interfering RNAs (siRNA), microRNAs (miRNA), and anti-sense nucleic acid.

In another aspect, the invention provides methods for identifying agents that are capable of restoring proper folding and function of ΔF⁵⁰⁸ CFTR. These methods involve (a) synthesizing one or more structural analogs of a lead compound selected from the group consisting of N-ω-acetylhistamine and 1-benzylimidazole, and (b) performing a functional assay on the analogs to identify an analog that has an improved biological or pharmaceutical property relative to that of the lead compound. These methods enable identification of agents that are capable of restoring proper folding and function of ΔF⁵⁰⁸ CFTR. In some of these methods, the improved biological or pharmaceutical property is an enhanced activity in inhibiting QPCT enzymatic activity.

A further understanding of the nature and advantages of the present invention may be realized by reference to the remaining portions of the specification and claims.

### DESCRIPTION OF THE DRAWINGS

FIG. 1, Rescue of ΔF508 CFTR channel function defect by knockdown of ΔF508 CFTR interactors in human primary CF bronchial epithelial and CFBE41o-cells. a. Experimental schematic. Primary bronchial epithelial cells or CFBE41o-cells were infected with lentiviral shRNAs before seeding onto snapwells, culturing the cells at air-liquid interface (ALI) for 28-30 days and measuring short circuit current in an Using chamber. b. Representative traces of forskolin (10 μM) and genistein (50 μM) activated ΔF508 CFTR short circuit current (I_{sc}). c. Quantification of the peak CFTR Inhibitor 172 (Inh 172)-sensitive I_{sc} (ΔI_{sc}) In CFBE41o-cells (n=3-5) and in human primary CF bronchial epithelial cells (DHBE, n=2 to 5) after knockdown of the indicated interactors as fold change relative to non-target shRNA (NT sh). Data represent mean±s.e.m.

FIG. 2, CoPIT workflow and results, a. Schematic overview of the Co-PIT workflow. Top panel: Cell lysates for Immunoprecipitation were prepared from ≥4×10⁷ lung epithelial cells (CFBE41o- or HBE41o-) with emphasis on extracting both cytoplasmic and membrane protein interactors of CFTR and pre-cleared before Co-IP with anti-CFTR antibody 3G11. Proteins eluted from the beads were purified by methanol/chloroform precipitation and digested with trypsin, before loading onto a MudPIT column and online MudPIT data acquisition. Lower Panel: Resulting spectra were searched with ProLuCID and search results filtered with DTASelect 2.1 to a protein false positive rate of <1% before normalization and further statistical analysis of the dataset. Core CFTR interactomes were determined by modeling the distribution functions of control and sample IPs, and applying corresponding confidence scores and abundance filters. Corresponding networks were graphed using the Radial Topology Viewer and differential comparison carried out. Data is stored in the Proteomics INTegrator (PINT) tool. b. Improved recovery of CFTR and interactors. Western blot depicting improved recovery of ΔF508 CF FR from CFBE41o-cells with TNI in comparison with different lysis buffers. A, B, and C indicate the different CFTR glycoloints. c. Western blot showing enhanced recovery of ΔF508 CFTR from beads after Co-IP with detergent and heat aided low pH elution in comparison to other directly mass spectrometric compatible elution methods. Lane “Wang et al. 2006”: Elution conditions as described in Wang et at, Cell 127, 803-815, 2006. Gly: Glycine. d. Enhanced sensitivity of the CFTR Co-IP and chromatography is reflected by enhanced spectral counts for CFTR itself and well-established interactors like HSP70 and HSP90. e, Comparison between the CFTR interactome reported by Wang et al. (Cell 127, 803-815, 2006) and this study. 33 of the reported 38 interactions in Calu-3 cells were recovered; 20 were confirmed as highly confident interactions (innermost circle) and 13 as medium confident interactions in this study, achieving an almost complete overlap of the two datasets. f. The table shows the recovery of CFTR and exemplary, well-characterized interactors in Co-IPs of wt CFTR (BHK cells (from Wang et al., Cell 127, 803-815, 2006) or HBE41o-cells (this study). g. Frequency distribution Nᵣ_{_{p ec}} of all r_{p ec} determined for the experimental condition wt CFTR to control condition. Individual points (black dots) indicate the individual vᵣ_{_{p ec}} values. The two-terra Gaussian fit is shown in grey. The individual Gaussian describing the distribution of non-specific binding is indicated by the left peak, whereas the Gaussian describing the enrichment for weak specific interactors is indicated by the right peak. The black arrow marks the r_{p ec} determined for CFTR, the bait protein. Right panel: Example P-values for well-known CFTR interactors (top four lines) and proteins commonly identified as background in Co-IP experiments (bottom 5 lines). Threshold for a high-confidence ΔF508-CFTR interactor was calculated at ≥0.92.

FIG. 3. CFTR interactome and validation of novel interactors. a. Western blotting of CFTR-IPs confirms specific interaction of CFTR with the novel interaction partners TRIM21, LGALS3BP and PTPLAD1 in CFBE41o- or HBE41o-cells. Results indicate similar binding of wt and ΔF508 CFTR with TRIM21 and LGALS3BP, and confirm enhanced binding of PTPLAD1 with ΔF508 CFTR. b. CFTR Co-IPs confirm CFTR interaction with TRIM21, PTPLAD1 and LGALS3BP in CF patient primary lung epithelial cells carrying either the ΔF508 or the F508S mutation. Control: CFTR null CFBE41o-cell line. c. Ubiquitin (UBB/UBC) recovery is increased in ΔF508 CFTR Co-IPs. d. CoPIT confidence scores and observed fold changes for TRIM21, LGALS3BP and PTPLAD1 match recovery in the IP-western blot. e, Reciprocal Co-IP using newly identified, endogenous interactors as bait confirms interaction of TRIM21, LGALS3BP and PTPLAD1 with ΔF508 CFTR and confirms differential binding of PTPLAD1 to wt and ΔF508 CFTR. Control, null; CFIR null CFBE41o-cell line, mock: beads only IP with no antibody added.

FIG. 4. Overview of drug treatment, siRNA mediated knockdown and temperature shift experiments. a. Schematic showing the experimental outline. b, Expression of different heat shock proteins. The Western blot shows expression of HSP90 (encoded by HSP90AA1 and HSP90AB1), GRP78 (HSPA5), GRP94 (HSP90B1) and HSP70 (HSPA1) during temperature shift to 30° C. c. CID MS2 spectrum of the acetylated HSPA1 peptide LDK(42.01)AQIHDLVLVGGSTR acquired on an Orbitrap XL. Mass shift of 42.01 indicates acetylation of HSP70 at Lysine 328 in the ATPase domain. Depicted are b- and y-fragment ions with the corresponding m/z values. All data are from independent biological replicates, wt (n=7), ΔF508 (n=8), SAHA (n=4), TSA (n=4), HDAC7 Cmpd 4a (n=3). d. Hierarchical clustering analysis of the CFTR core interactomes shows that the ΔF508 CFTR interaction profile clusters with high significance with those of ΔF508 CFTR at 1 h and 6 h temperature shifts to 30° C. (mutant cluster), whereas temperature shift to 30° C. for 24 h and temperature shift to 30° C. for 24 h with reversal cause the respective ΔF508 CFTR interaction profiles to significantly cluster with that of wt CFTR. Bootstrap values (10,000 samplings) are given for each tree node. Significant (bootstrap value >90) and highly significant clusters (bootstrap value >95) are shown on the dendrogram. The heatmap indicates the relative protein abundance values measured by mass spectrometry as negative log₁₀ ratios of interactors relative to CFTR. White in the heatmap indicates that no interaction was observed.

FIG. 5. Interaction profiles of proteins selected for the RNAi screen. a. Observed interaction profiles of selected candidates and CFTR (lower panel) and expected candidate profiles (upper panel). Norm. Int: normalized intensity. Ser.: scrambled. b. Lentiviral infection rates were greater than 97% after 48 h in CFBE41o-cells as indicated by control GFP infection.

FIG. 6. Western blot detection of ΔF508 CFTR upon RNAi of interactors. ΔF508 CFTR was detected 48-72 h after lentiviral shRNA infection using the 3G11 antibody or 24.1. antibody (lowest left panel). Rescue is indicated by appearance of band C. Detection of β-actin served as loading control. Samples on the same blot represent parallel infections. Samples in the lower three left panels were lysed initially in TNI buffer, whereas samples in the other panels were lysed directly in 2× Laemmli sample buffer as described in Materials and Methods. Ser: scrambled non-target shRNA.

FIG. 7, Co-localization of novel interactors with ΔF508 CFTR. a. Each panel contains immunofluorescence staining of CFTR, interactor as indicated, nuclei (DAP1) and the merged picture. Scale bars, 10 μm. b. Wt and ΔF508 CFTR was detected by immunofluorescence staining in HBE41o- and CFBE410-cells, respectively. Arrows points to wt CFTR at the plasma membrane of control cells. c. Schematic of a cell depicting sequential (spatio-temporal) regulation of ΔF508 CFTR protein biogenesis by the interactors targeted in the shRNA screen. Functional classification of interactors is indicated. Proteins detected in co-localization studies are marked in bold.

FIG. 8. ΔF508 CFTR detection in primary bronchial epithelial cells upon RNAi of key interactors. a. Quantification of the ΔF508 CFTR ion channel activity (as fold change of the ΔI_{sc} relative to non-target shRNA) in comparison to the ratio of band C to band A/B in primary CF patient or healthy donor (wt) cells. b. Representative trace of forskolin (10 μM, F) and genistein (50 μM, G) activated, wt CFTR short circuit current (I_{sc}) in a 30 d ALI culture from a healthy donor. CFTR inhibitor 172 (I) indicates specificity of the measured I_{sc} for CFTR. c. Western blot of 28-30 d old primary human bronchial epithelial snapwell cultures from CF patients (DHBE) indicates formation of band. C after specific knockdown of PABPC1, YBX1, PTBP1, TRIM21, PTPLAD1 and SURF4 with different shRNAs. Tubulin, β-actin or Na⁺/K⁺ATPase was used as a loading control. Knockdown of PABPC1 and PTPLAD1 was verified by Western blotting with the respective antibodies. NT sh: non-target shRNA.

FIG. 9. Halide assay results for CFTR chloride channel activity in stable cell clones, a. CFTR chloride channel activity was measured in HBE41lo-, CFBE41o- and CFBE410-cells with stable knockdown of LGALS3BP (clone 13) or PTPLAD1 (clone 24). Activity was measured by NaI mediated quenching of a halide sensitive Venus YFP. Time-lapse experiments show the Iodide influx following pre-incubation of cells with 50 μM Genistein. Additional stimulation with Forskolin was performed 15 s following addition of NaI. Representative single cell traces are shown. Inset shows the fitted fluorescence decay time constant for each trace. b. Western blot showing the negative influence of LGALS3BP knock down on ΔF508 CFTR protein stability. Clone 13.1 and 13.2 are two independent CFBE41o-clones that stably express an shRNA against LGALS3BP. The knockdown was validated by detection of LGALS3BP. c. Western blot showing increased production of ΔF508 band C in CFBE41o-cell clone 24 stably expressing an shRNA against PTPLAD1. The knock down was validated by detection of PTPLAD1. Detection of β-actin served as loading control. Scr: scrambled non-target shRNA.

FIG. 10. Percentage of CFTR interactors associated with known protein misfolding and other prevalent diseases. The bar graph shows the fraction of the interactome associated with genetic diseases listed in OMIM. Percentages next to the disease name indicate the percentage of ΔF508 CFTR specific interactors involved in these diseases. Interactors causative for Alzheimer disease and other neurodegenerative diseases like Leigh-syndrome are enriched in the ΔF508 CFTR interactome. “Other” indicates diseases not fitting into one of the other categories listed.

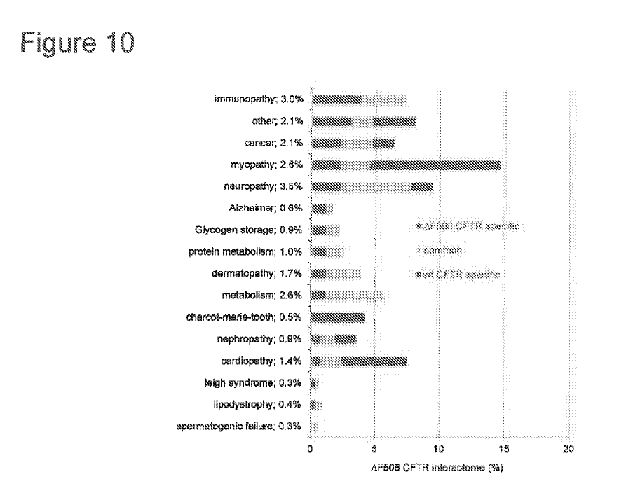

FIG. 11. Activities of QPCT small molecule inhibitors on restoring ΔF508 CFTR function. a. Time course treatment of CFBE cells expressing ΔF508 CFTR with N-ω-acetylhistamine (NAH, dissolved in DMSO) at two different concentrations (100 or 200 μM). DMSO was used as a vehicle control. After 48 hours of treatment, Band C becomes visible, and after 72 h or 6 days of treatment the band C becomes more pronounced and the ratio of Band C to Band A/B increases. Treatment with 5 μM VX809 was used as a positive control. b. Fold change in ΔF508 CFTR channel activity after treatment of CFBE cells with different inhibitors for 24 h (indicated in text) and as measured in Ussing chamber experiments.

### DETAILED DESCRIPTION

### 1. Introduction

It is known that ΔF508 CFTR function can be partially rescued by a shift to low temperature (26 to 30° C.) or inhibition of histone deacetylase (HDAC) activity. However, it is largely unknown which processes or interactions lead to stabilization and partial restoration of channel activity of ΔF508 CFTR observed upon shift to permissive temperature or upon inhibition of HDAC activity. The present invention is based in part on the studies undertaken by the present inventors to better understand the disease causing events and to establish a molecular basis for therapeutic targeting. Specifically, the inventors performed a systematic and comprehensive analysis of the normal and ΔF508 CFTR interactome in patient-derived lung epithelial cells and its dynamics during temperature shift and HDACi. A comprehensive interactome coverage was achieved by using a novel deep proteomic analysis method based on co-immunoprecipitation-mass spectrometry (CoPIT). The inventors identified more than 21,000 spectra for CFTR alone and found a total of 638 individual high-confidence interactors with 208 specific for ΔF508 CFTR. These constitute a mutation-specific interactome, which is extensively remodeled upon rescue by temperature-shift, HDACi or RNAi of HDAC7.

Studies of the inventors suggest that the loss of ΔF508 CFTR function emerges from novel associations with multiple alternate protein complexes and cellular pathways that route ΔF508 CFTR differently than wt CFTR. Importantly, network-analysis of the dynamic interactome identified a number of key novel interactors that influence translation and degradation of ΔF508 CFTR in particular, as well as its ER-dependent folding and trafficking upon rescue. Some of these were functionally tested and were found to alter ΔF508 CFTR maturation, among them the ER-resident protein tyrosine kinase-like protein PTPLAD1 and the protein disulfide isomerase PDIA4, the translation regulating protein YBX1 and the ubiquitin E3-ligase TRIM21. Some proteins are critical for normal CFTR biogenesis and include the lectin binding protein LGALS3BP whose loss leads to elimination of CFTR protein. The results described herein demonstrate that global remodeling of protein interactions involved in CFTR biogenesis and degradation is crucial for ΔF508 CFTR rescue, and identify individual key ΔF508 CFTR interactors like PTPLAD1 or SURF4 whose loss enhanced ΔF508 CFTR channel function. The studies also show that reduction of protein levels of some of the ΔF508 CFTR interactors identified herein rescues channel function of ΔF508 CFTR. Thus, as exemplified herein, modulation of these interactors provides a promising route to correction of the ΔF508 CFTR defect.

In accordance with these discoveries, the invention provides methods for restoring normal CFTR function in cells harboring the ΔF508 CFTR mutant. The invention also provides novel methods for treating cystic fibrosis. Preferably, subjects suitable for treatment with the methods of the invention are those with the ΔF508 CFTR mutation. Some of the methods employ the small molecule compounds described herein and their derivatives for restoring physiological activities of ΔF508 CFTR in vivo and for treating cystic fibrosis in patients. Some other therapeutic methods of the invention can employ an agent (e.g., a small inhibitory oligonucleotide) that downregulate the expression of the ΔF508 CFTR interactor proteins or inhibits their interaction with ΔF508 CFTR. Also provided in the invention are methods for identifying additional compounds capable of restoring ΔF508 CFTR function. Some of these methods are directed to identifying compounds that are derivatives of the specific small molecule inhibitors of glutaminyl-peptide cyclotansferase (QPCT) described herein. Some other methods are directed to identifying compounds that can inhibit or suppress ΔF508 CFTR interaction with the interactor proteins described herein,

The following sections provide more detailed guidance for making and using the compositions of the invention, and for carrying out the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Illustrated Dictionary of Immunology, Cruse (Ed.), CRC Pr 1 LIc (2^{nd} ed., 2002); Oxford Dictionary of Biochemistry and Molecular Biology, Smith et al. (Eds.), Oxford University Press (revised ed., 2000); Encyclopaedic Dictionary of Chemistry, Kumar (Ed.), Ammol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” or “candidate agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein. In some screening methods of the invention, the employed candidate agents or candidate compounds are small organic molecules.

The term “analog” or “derivative” is used herein to refer to a molecule that structurally resembles a reference molecule (e.g., a ΔF508 CFTR interactor-inhibiting compound exemplified herein) but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs to identify variants of known compounds having improved traits (such as higher binding affinity for a target molecule) is an approach that is well known in pharmaceutical chemistry.

As used herein, the cellular activity of a protein (e.g., a ΔF508 CFTR interactor) refers to the biological function that is naturally performed by the protein in the wildtype cellular environment where the protein is expressed. Thus, depending on the specific biochemical nature of the protein, the cellular activity can be; e.g., an enzymatic activity, transcriptional regulatory activity, forming cellular structures, signal transducing activity, activity in transport and storage, and activity in cell proliferation, differentiation or cell death.

Cystic fibrosis is an inherited disease of the secretory glands which mainly affects the lungs, pancreas and liver. Currently there is no cure. Cystic fibrosis is by caused a mutation in the gene coding for the cystic fibrosis transmembrane conductance regulator (CFTR) polypeptide. CFTR polypeptide is an ion channel that transports chloride and thiocyanate across the epithelial cell membrane and hence acts to regulate components of sweat, digestive juices and mucus. Respiratory dysfunction is the most serious symptom and results from frequent lung infections. Most individuals die in their 20s and 30s from lung failure and lung transplantation is often necessary as CF worsens. A multitude of other symptoms, including sinus infections, poor growth, diarrhea and infertility result from the effects of CF on other parts of the body.

Cystic fibrosis is a recessive genetic disorder. Sequence analysis of the CFTR gene of CF chromosomes has revealed a variety of disease causing mutations. To date, >1000 disease causing mutations in the CFTR gene have been identified. The most common mutation results in the deletion of phenylalanine in the ATP binding cassette at position 508 of the CFTR protein. The resultant ΔF⁵⁰⁸ CFTR polypeptide is expressed as a large, misfolded nascent polypeptide which is prematurely destroyed via the ubiquitin pathway but which aggregates following defective processing in the translocation machinery.

The coding regions of CFTR axe composed of 27 exons dispersed over 250,000 base pairs (250 Kb) of genomic DNA. During transcription, introns are spliced out and exons are joined together to form a 6100-bp mRNA transcript that is translated into the 1480 amino acid sequence of CFTR protein. The normal CFTR protein is a chloride channel protein found in membranes of cells that line the passageways of the lung, pancreas, colon and genitourinary tract. The CFTR protein is made up of five domains: two membrane-spanning domains (MSD1 and MSD2) that form the chloride ion channel, two nucleotide-binding domains (NBD1 and NBD2) that hind and hydrolyze adenosine triphosphate (ATP), and a regulatory (R) domain. The deletion of phenylalanine residue 508 in the first nucleotide-binding domain (NBD1) results in retention of the mutant protein in the endoplasmic reticulum (ER) and subsequent degradation by the ubiquitin-proteosome pathway.

Cystic fibrosis transmembrane conductance regulator” (or “CFTR”) gene or nucleic acid as used herein is known and described in, for example, U.S. Pat. No. 6,201,107 and U.S. Pat. No. 5,776,677. The CFTR protein is described in, for example, U.S. Pat. No. 5,543,399. The CFTR nucleic acid and protein is further described in NCBI Number NM 000492 and NCBI Number P13569. CFTR nucleic acids and proteins that may be employed in the practice of the present invention are generally mammalian (e.g., dog, cat, mouse, rat, rabbit) and preferably human. The CFTR protein has six extra-cytoplasmic loops designated EL1 through EL6. CFTR as used herein may be a wild-type (e.g., contains phenylalanine at position 508) or a mutated CFTR that would produce disease in a subject containing such a mutation (e.g., a deletion of phenylalanine at position 508 or other mutations).

Administration “in conjunction with” one or more other therapeutic agents includes simultaneous (concurrent) and consecutive administration in any order.

The term “contacting” has its normal meaning and refers to combining two or more agents (e.g., polypeptides or small molecule compounds) or combining agents and cells. Contacting can occur in vitro, e.g., combining two or more agents or combining an agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

The terms “subject” and “patient” are used interchangeably and refer to mammals such as human patients and non-human primates, as well as experimental animals such as rabbits, rats, and mice, and other animals. Animals include all vertebrates, e.g., mammals and non-mammals, such as dogs, cats, cheeps, cows, pigs, rabbits, chickens, and etc. Preferred subjects for practicing the therapeutic methods of the present invention are human.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. Compounds Capable of Restoring Function of ΔF508 CFTR

The invention provides agents that can restore the proper folding and biological activities (e.g., incorporation into the plasma membrane and channel activity) and can promote formation of the fully glycosylated form of ΔF508 CFTR (band C) of the ΔF508 CFTR mutant cystic fibrosis transmembrane conductance regulator (CFTR), which is the cause for a majority of cystic fibrosis cases. The present inventors identified novel proteins in the CFTR protein interaction network (interactome) that contributes to the misfolding and loss of function of ΔF508 CFTR. Molecules in CFTR interactome include interactor proteins (“interactors”) that directly or indirectly interact with CFTR. It was found that the ΔF508 mutation leads to derailment of the CFIR interactome by mainly recruiting some novel, disease-specific interactors. As detailed in the Examples herein, a number of such ΔF508 CFTR interactors were identified from ΔF508 CFTR mutation-specific interactome. These include, e.g., polyadenylate-binding protein 1 (PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), E3 ubiquitin-protein ligase TRIM21 (TRIM21), protein-tyrosine phosphatase-like A domain-containing protein 1 (PTPLAD1), protein-glutamine gamma-glutamyltransferase 2 (TGM2), RelA-associated inhibitor (PPP1R13), and glutaminyl-peptide cyclotransferase (QPCT). In proof of principle experiments, functional inhibition or expression knockdown of these interactor proteins (e.g., via shRNA) was performed in primary CF patient bronchial epithelia as well as in epithelia generated from the CT model cell line CFBE41o. This resulted in enhanced proper folding of ΔF508 CFTR as determined by its glycosylation pattern (Western blot) and partially restored ΔF508 CFTR chloride channel function as determined by electrophysiology.

In accordance with the invention, any agents (e.g., inhibitory polynucleotides or small molecule compounds) capable of modulating function or abundance of the specific ΔF508 CFTR interactors disclosed herein (called “drug target proteins”) may be used for treating Cystic Fibrosis via rescuing channel function of ΔF508 CFTR. These include compounds that can modulate the target proteins in their abundance by means of modification of genetic sequence (genomic DNA), mRNA products thereof, the amount of mRNA product, the mRNA's availability for translation, the amount of the protein itself, or modulation of the target protein's function by means of modification of the ability to bind to, modify, or get released from additional proteins, or any small molecule targeted against the interacting protein or its mechanistic function with the aim to treat Cystic Fibrosis. In various embodiments, therapeutic methods of the invention can employ agents that downregulate the expression of the specific ΔF508 CFTR interactor proteins described herein, agent that specifically inhibit the biological activities (e.g., enzymatic activities) of the interactor proteins, or agents that suppresses or disrupt ΔF508 CFTR interaction with these proteins (e.g., antibodies targeting these proteins).

Some embodiments of the invention provide inhibitory polynucleotide molecules that specifically target the ΔF508 CFTR interactor proteins and restores the channel function of ΔF508 CFTR. Examples of inhibitory polynucleotides include, e.g., short interfering RNAs (siRNAs), microRNAs (miRNAs), short hairpin RNAs (shRNAs), anti-sense nucleic acids, and complementary DNAs (cDNAs). Such inhibitory polynucleotides (e.g., siRNA or shRNA) can be employed to rescue the normal biological function ΔF508 CFTR, in cells harboring the mutant protein or to treat subjects afflicted with CF caused by ΔF508 CFTR. As demonstrated herein in the Examples below, normal channel function in ΔF508 CFTR can be rescued by RNAi against any of the CFTR interactor proteins, including QPCT, PABPC1, YBX1, PTBP1, SURF4, PDIA4, TRIM21, PTPLAD1, TGM2, and ReIA-associated inhibitor (PPP1R13). Specifically, it was shown knockdown of some single interactors, e.g., YBX1, PTPLAD1 or PABPC1, was sufficient to induce partial rescue and enhance ΔF508 CFTR channel function.

Using inhibitory polynucleotide molecules to specifically silence expression of a target gene has been well known and routinely practiced in the art. Such nucleic acid agents that specifically target a ΔF508 CFTR interactor protein can be prepared using methods well known in the art. Interference with the function and expression of endogenous genes by double-stranded RNA has been shown in various organisms such as C. elegans as described, e.g., in Fire et al, Nature 391:806411, 1998; drosophilia as described, e.g., in Kennerdell et al., Cell 95:1017-4026, 1998; and mouse embryos as described, e.g., in Wianni et al., Nat. Cell Biol. 2:70-75, 2000. In general, such double-stranded RNA can be synthesized by in vitro transcription of single-stranded RNA read from both directions of a template and in vitro annealing of sense and antisense RNA strands. Double-stranded RNA can also be synthesized from a cDNA vector construct in which a target gene is cloned in opposing orientations separated by an inverted repeat. Following cell transfection, the RNA is transcribed and the complementary strands reanneal. Double-stranded RNA targeting the target gene can be introduced into a cell by transfection of an appropriate construct. The inhibitory polynucleotides targeting the ΔF508 CFTR interactor proteins can all be obtained commercially or synthesized via routinely practiced techniques of molecular biology. By way of example, a number of shRNAs targeting several ΔF508 CFTR interactor proteins and their corresponding target sequences are described in the Examples below.

In some other embodiments, the invention can utilize small molecule compounds or other inhibitors that specifically suppress the normal cellular activity or biological function of the CFTR interactors described herein. For example, the inventors discovered that several specific small molecule inhibitors of glutaminyl-peptide cyclotransferase (aka “glutamyl cyclase”) QPCT are able to increase ΔF508 CFTR channel function in vitro. These small molecule inhibitors include, e.g., N-ω-acetylhistamine, 1-benzylimidazole, and their functional derivatives and analogs. These compounds restore ΔF508 CFTR channel function presumably via by targeting the enzymatic activity of QPCT. Thus, some specific embodiments of the invention are directed to using these QPCT inhibitors or functional analogs to rescue the normal biological activities of ΔF508 CFTR and to treat subjects suffering from cystic fibrosis. Some other methods of the invention rely on agents that suppress or inhibit ΔF508 CFTR interaction with the other interactor proteins identified herein.

As exemplified herein, small molecule compounds such as N-ω-acetylhistamine (systematic name: N-[2-(1H-Imidazol-4-yl)ethyl]acetamide) and 1-benzylimidazole (systematic name: 1 -Benzyl-1H-imidazole) can enhance ΔF508 CFTR channel function presumably by inhibiting the enzymatic function of QPCT. These compounds can be readily employed in the therapeutic methods of the invention. In addition to the QPCT inhibitor compounds exemplified herein, derivative compounds that can be easily synthesized from these compounds may also be suitable for the practice of the methods of the invention. For example, relative to N-ω-acetylhistamine or 1-benzylimidazole, some of their derivative compounds can have one or more mono- or multi-valent groups replaced with a different mono- or multi-valent group. The replaced group can be, e.g., H; halogen; straight, cyclic or branched chain alkyl; straight, cyclic or branched chain alkenyl; straight, cyclic or branched chain alkynyl; -alkenyl or -alkynyl; CN; CF₃; aryl and substituted aryl groups in which any or all H groups of the aryl ring is substituted with a different group; heterocyclic and substituted heterocyclic groups in which any or all groups of the aryl ring is substituted with a different group; carboxyl; carbonyl, alkoxyl; alkyloxyalkanes; alkoxycarbonyl; aryloxyl, heterocyclyloxyl; hydroxyl; amine; amide; amino; quaternary amino; nitro; sulfonyl; alkylamine; silyl, siloxyl; saturated C—C bonds; unsaturated C—C bonds; ester, ether, amino; amide, urethane, carbonyl, acetyl and ketyl groups; hetero atoms, including N, S and O; polymer groups; and amino acids. In some derivative compounds, one or more hydrogens can he substituted with a lower alkyl group. The various derivative compounds can be subject to a functional test (e.g., proliferation inhibition assay as exemplified herein) to ascertain their CFTR interactor-inhibiting activities.

In some embodiments, variants or derivative compounds with similar or improved properties can be obtained by rational optimization of the exemplified QPCT inhibitor compounds (the lead compounds). Optionally, the compounds generated via rational design can be further subjected to a functional test or screening in order to identify compounds with improved activities. Detailed methods for designing and screening such variant compounds are described below. The various QPCT inhibitor compounds exemplified herein and their variants or derivatives can all be readily obtained from commercial suppliers (e.g., Maybridge, Cornwall, England) or de novo synthesized using routinely practiced methods of organic chemistry.

### Identifying Novel ΔF508 CFTR-Modulating Compounds

The ΔF508 CFTR-interacting proteins (or “interactor proteins”) described herein provide novel targets to screen for compounds that can suppress or downregulate these disease-specific interactors and for rescuing ΔF508 CFTR function. These novel targets include, e.g., glutaminyl-peptide cyclotransferase (QPCT), polyadenylate-binding protein 1 (PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), E3 ubiquitin-protein ligase TRIM21 (fRIM21), protein-tyrosine phosphatase-like A domain-containing protein I (PTPLAD1), protein glutamine gamma-glutamyltransferase 2 (TGM2), and RelA-associated inhibitor (PPP1R13). The invention accordingly provides methods for identifying compounds that suppress the expression of these novel targets or that inhibit the biological activities of these target proteins. Various biochemical and molecular biology techniques or assays well known in the art can be employed to practice the screening methods of the present invention. Such techniques are described in, e.g., Handbook of Drug Screening, Seethala et. al. (eds.), Marcel Dekker (1^{st} ed., 2001); High Throughput Screening: Methods and Protocols (Methods in Molecular Biology, 190), Janzen (ed.), Humana Press (1^{st} ed., 2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003). Further guidance to practice the screening methods of the present invention is provided below.

Typically, candidate agents or compounds are first assayed for ability to modulate (e.g., inhibit) a biological activity or to downregulate expression or cellular level of a ΔF508 CFTR-interacting protein described herein (e.g., QPCT or PDIA4) (“the first assay step”). Modulating compounds thus identified are then subject to further screening for ability to restore proper folding of ΔF508 CFTR or channel activity, typically in a cell that harbors ΔF508 CFTR (“the second testing step”). Depending on the specific interactor protein employed in the method, modulation of different biological activities of the ΔF508 CFTR-interacting proteins can be assayed in the first step. For example, the candidate agents can be screened for ability to modulate a known biochemical or enzymatic function of the ΔF508 CFTR-interacting protein. The candidate agents can be assayed for activity to modulate expression or cellular level of the ΔF508 CFTR-interacting protein, e.g., its transcription or translation. The candidate agents can also be screened for modulating (e.g., inhibiting or disrupting) the association between the interactor and ΔF508 CFTR.

In some preferred embodiments, the ΔF508 CFTR-interacting protein employed in the screening methods is an enzyme (e.g., QPCT). In these methods, the biological activity monitored in the first screening step can be the specific enzymatic activity of the ΔF508 CFTR-interacting protein. The candidate agents are typically screened for ability to down-regulate a biological activity or expression level of the interactor protein in the first assay step. Once candidate agents that modulate the interactor protein are identified, they are typically further tested for ability to modulate ΔF508 CFTR cellular function, e.g., facilitating its full glycosylation or restoring its channel function. This further testing step is often needed to confirm that their modulatory effect on the ΔF508 CFTR-interacting protein would indeed lead to rescue or restoration of the normal cellular activities of ΔF508 CFTR. Restoration of the cellular activities of ΔF508 CFTR can be readily assessed via standard assays routinely practiced in the art, e.g., glycosylation pattern analysis or the Ussing Chamber electrophysiological assay as exemplified herein.

In both the first assaying step and the second testing step, either an intact interactor protein, or a fragment thereof, may be employed. Analogs or functional derivatives of the ΔF508 CFTR-interactor protein could also be used in the screening. The fragments or analogs that can be employed in these assays usually retain one or more of the biological activities of the interactor protein (e.g., enzyme activity if the interactor protein employed in the first assaying step is an enzyme). Fusion proteins containing such fragments or analogs can also be used for the screening of candidate agents. Functional derivatives of an interactor protein usually have amino acid deletions and/or insertions and/or substitutions while maintaining one or more of the bioactivities and therefore can also be used in practicing the screening methods of the present invention. A functional derivative can be prepared from an interactor protein by proteolytic cleavage followed by conventional purification procedures known to those skilled in the art. Alternatively, the functional derivative can be produced by recombinant DNA technology by expressing only fragments of a ΔF508 CFTR-interactor protein that retain one or more of their bioactivities.

Candidate agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some candidate agents are synthetic molecules, and others natural molecules. Candidate agents can be obtained from a wide variety of sources including libraries of synthetic or natural compounds. Combinatorial libraries can be produced for many types of compound that can be synthesized in. a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH -3 domains, serines, threonines, tyrosines or histidines for phosphorylation sites, or to purines.

The candidate agents can be naturally occurring proteins or their fragments. Such candidate agents can be obtained from a natural source, e.g., a cell or tissue lysate. Libraries of polypeptide agents can also be prepared, e.g., from a cDNA library commercially available or generated with routine methods. The candidate agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the candidate agents are polypeptides or proteins. The candidate agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

In some preferred methods, the candidate agents are small molecule organic compounds, e.g., chemical compounds with a molecular weight of not more than about 1,000 or 500. Preferably, high throughput assays are adapted and used to screen such small molecules, In some methods, combinatorial libraries of small molecule candidate agents as described above can be readily employed to screen for small molecule compound inhibitors of ΔF508 CFTR-interactor protein that is an enzyme (e.g., QPCT). Depending on the interactor protein used, a number of assays for the screening can be developed based on the biochemical nature of the interactor protein and screening format well known in the art. See, e.g., Schultz (1998) Bioorg Med Chem Lett 8:2409-2414; Weller (1997) Mol Divers. 3:61-70; Fernandes (1998) Curr Opin Chem Biol. 2:597-603; and Sittampalam (1997) Curl Opin Chem Biol 1:384-91.

ΔF508 CFTR-rescuing modulator compounds of the present invention also include antibodies that specifically bind to a ΔF508 CFTR-interactor protein described herein. Such antibodies can be monoclonal or polyclonal. Such antibodies can be generated using methods well known in the art. For example, the production of non-human monoclonal antibodies, e.g., murine or rat, can be accomplished by, for example, immunizing the animal with a ΔF508 CFTR-interactor protein or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression. Humanized forms of mouse antibodies can be generated by linking the CDR regions of non-human antibodies to human constant regions by recombinant DNA techniques. See Queen et al., Proc, Natl. Acad. Sci. USA 86, 10029-10033 (1989) and WO 90/07861, Human antibodies can be obtained using phage-display methods. See, e.g., Dower et al., WO 91/17271; McCafferty et at, WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a ΔF508 CFTR-interactor protein. Activities of the antibodies in restoring ΔF508 CFTR function can be similarly examined with the assays described herein.

### V. Screening for Optimized QPCT Inhibitors for Restoring ΔF508 CFTR Function

In addition to the small molecule QPCT inhibitor compounds exemplified herein and variants discussed above, the invention also provides methods of screening for additional small molecule compounds that are derived from these specific QPCT inhibitors. Some of the screening methods of the present invention are directed to identifying analogs or derivatives of the exemplified QPCT inhibitors with improved properties. An important step in the drug discovery process is the selection of a suitable lead chemical template upon which to base a chemistry analog program. The process of identifying a lead chemical template for a given molecular target typically involves screening a large number of compounds (often more than 100,000) in a functional assay, selecting a subset based on some arbitrary activity threshold for testing in a secondary assay to confirm activity, and then assessing the remaining active compounds for suitability of chemical elaboration.

Some of the screening methods employ one of the specific QPCT inhibitors exemplified herein (e.g., N-ω-acetylhistamine and 1-benzylimidazole) as a lead compound to search for related compounds that have improved biological or pharmaceutical properties. For example, analogs or derivatives of these QPCT inhibitors can be screened for to identify compounds that have a stronger QPCT-inhibiting activity, e.g., a lower IC50 value determined from a spectrometric assay for glutaminyl cyclase activity (see, e.g., Schilling et al., Anal. Biochem, 303:49-56, 2002). Compounds with such improved properties can be more suitable for various pharmaceutical applications. The screening methods typically involve synthesizing analogs, derivatives or variants of a QPCT inhibitor (e.g., N-ω-acetylhistamine or 1-benzylimidazole). Often, a library of structural analogs of a given QPCT inhibitor is prepared for the screening. A functional assay (e.g., glutaminyl cyclase activity inhibition as described herein) is then performed to identify one or more of the analogs or derivatives that have an improved biological property relative to that of the QPCT inhibitor from which the analogs or variants are derived. Compounds with improved inhibitory activity on QPCT can be further screened for ΔF508 CFTR-rescuing ability or for improved pharmaceutical properties.

Structures and chemical properties of the lead QPCT inhibitors have been characterized in the art. To synthesize analogs or derivatives based from the chemical backbones of these QPCT inhibitors, only routinely practiced methods of organic chemistry that are well known to one of ordinary skill in the art are required. For example, combinatorial libraries of chemical analogs of a known compound can be produced using methods described herein. Exemplary methods for synthesizing analogs of various compounds are described in, e.g., Overman, Organic Reactions, Volumes 1-62, Wiley-Interscience (2003); Broom et al., Fed Proc. 45: 2779-83, 1986; Ben-Menahem et al., Recent Frog Horm Res. 54:271-88, 1999; Schramm et al:, Annu. Rev, Biochem, 67: 693-720, 1998; Bolin et al., Biopolymers 37: 57-66, 1995; Karten et al., Endocr Rev. 7: 44-66, 1986; Ho et al., Tactics of Organic Synthesis, Wiley-Interscience; (1994); and Scheit et al., Nucleotide Analogs: Synthesis and Biological Function, John Wiley & Sons (1980). Once a library of candidate structural analogs of a lead QPCT inhibitor compounds are synthesized, a functional assay is then performed to identify one or more of the analogs or derivatives that have an improved biological property relative to that of the lead compound. The desired compound may have an improved property (e.g., a reduction of IC₅₀ as measured in a proliferation inhibition assay) that is at least 10%, 25%, 50%, 75%, 100%, 200%, or 500% better than that of the lead compound. Any assays known in the art for assessing QPCT biochemical activities can be used to identify an improved property in analogs or derivatives of a given QPCT inhibitor. These include the glutaminyl cyclase activity assay. Compounds identified from the assays can then be examined to ensure they possess the same or better ability in restoring ΔF508 CFTR cellular function as the lead compound. This can be performed by, e.g., examining the presence of fully glycosylated ΔF508 CFTR (Band C) with western blot or assessing ΔF508 CFTR channel activity with the Ussing chamber assay in a suitable cell line (e.g., CFBE41o-bronchial epithelial cell line) as exemplified herein. Alternatively or additionally, the identified derivative or analog compounds can be assayed to identify compounds with better pharmaceutical properties, e.g, enhanced in vivo half-life, Improved pharmaceutical properties of a QPCT inhibitor analog can be assayed using standard methods routinely practiced in the art, e.g., as described in, Remington: The Science and Practice of Pharmacy, Mack Publishing Co., 20^{th} ed., 2000. Compounds with such improved properties can be more suitable for various therapeutic applications described herein.

### VI. Therapeutic Applications

The ΔF508 CFTR-modifying compounds (or “ΔF508 CFTR interactor-inhibiting compounds”) described herein, including the small molecule QPCT inhibitors, their related derivative compounds, as well as other agents (e.g., small inhibitory polynucleotides such as shRNAs) targeting ΔF508 CFTR-interactor proteins, can find uses in various therapeutic applications. In some embodiments, the invention provides methods for employing these agents for restoring proper folding and channel function of ΔF508 CFTR in cells harboring the CFTR mutant. In some related therapeutic applications, the invention provides methods for treating or ameliorating symptoms in subjects suffering from cystic fibrosis. Typically, the therapeutic methods of the invention entail administering to a subject a pharmaceutical composition that comprises an effective amount of the therapeutic agent described herein (e.g., an inhibitory polynucleotide or a small molecule). Novel therapeutic agents that can be identified in accordance with the screening methods of the invention can also be employed.

The compounds can be administered alone to a subject in need of treatment. More preferably, they are administered in the form of a pharmaceutical composition or preparation in admixture with any of various pharmacologically-acceptable additives. For example, the compounds may be administered in the form of a convenient pharmaceutical composition or formulation suitable for oral, topical, parenteral application, or the like. Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lacticle polymer, lactide/glycolide copolymer, or polyoxyethylene-polyoxypropylene copolymers may be used to control the release of the compounds. Other potentially uselitl parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

Pharmaceutical composition containing a ΔF508 CFTR-modifying compound or CFTR interactor-inhibiting compound described herein can be administered locally or systemically in a therapeutically effective amount or dose. They can be administered parenterally, enterically, by inhalation, by injection, rapid infusion, nasopharyngeal absorption, dermal absorption, and orally. The inhibitors for use in the methods of the invention should be administered to a subject in an amount that is sufficient to achieve the desired therapeutic effect (e.g., eliminating or ameliorating symptoms associated with cystic fibrosis) in a subject in need thereof. Typically, a therapeutically effective amount or efficacious dose of the inhibitor compound employed in the pharmaceutical compositions of the invention should inhibit expression or activities of a CFTR interactor in a cell, or suppress or reverse CF symptoms in a subject. Actual dosage levels of the active ingredients in the pharmaceutical compositions of the present invention can be varied so as to obtain an amount of the active ingredient which is effective to achieve the desired therapeutic response without being toxic to the subject.

The ΔF508 CFTR-modifying compounds of the invention or pharmaceutical compositions containing the compounds can also be used in combination with known drugs or regimens for treating cystic fibrosis. Existing medications used to treat patients with cystic fibrosis include pancreatic enzyme supplements; multivitamins (including fat-soluble vitamins); mucolytics; nebulized, inhaled, oral, or intravenous antibiotics; bronchodilators; anti-inflammatory agents; agents to treat associated conditions or complications (e.g., insulin, bisphosphonates); and agents devised to potentially reverse the abnormalities in chloride transport (e.g., ivaraftor and lumacaftor/ivacaftor). In such combination therapy, the ΔF508 CFTR-modifying compounds of the invention can be administered to the patients sequentially (prior to or subsequent to) or simultaneously with the known treatment regimen.

The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. For a given ΔF508 CFTR-modifying compound (or “ΔF508 CFTR interactor-inhibiting compound”) described herein, one skilled in the art can easily identify the effective amount of an agent that inhibits a CFTR interactor by using routinely practiced pharmaceutical methods. Dosages used in vitro or in situ studies may provide useful guidance in the amounts useful for in vivo administration of the pharmaceutical composition, and animal models may be used to determine effective dosages for treatment of particular disorders. Typically, a pharmaceutically effective dosage would be between about 0.001 and 100 mg/kg body weight of the subject to be treated.

The ΔF508 CFTR-modifying compounds (e.g., QPCT inhibitor compounds) and other therapeutic regimens described herein are usually administered to the subjects on multiple occasions. Intervals between single dosages can be daily, weekly, monthly or yearly. Intervals can also be irregular as indicated by measuring blood levels of the QPCT inhibitor compounds and the other therapeutic agents used in the subject. In some methods, dosage is adjusted to achieve a plasma compound concentration of 1-1000 μg/ml, and in some methods 25-300 μg/ml or 10-100 μg/ml. Alternatively, the therapeutic agents can be administered as a sustained release formulation, in which case less frequent administration is required. Dosage and frequency vary depending on the half-life of the inhibitor compound and the other drugs in the subject. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

### EXAMPLES

The following examples are offered to illustrate, but not to limit the present invention.

### Example 1

### ΔF508 CFTR Mutation Specific Interactome

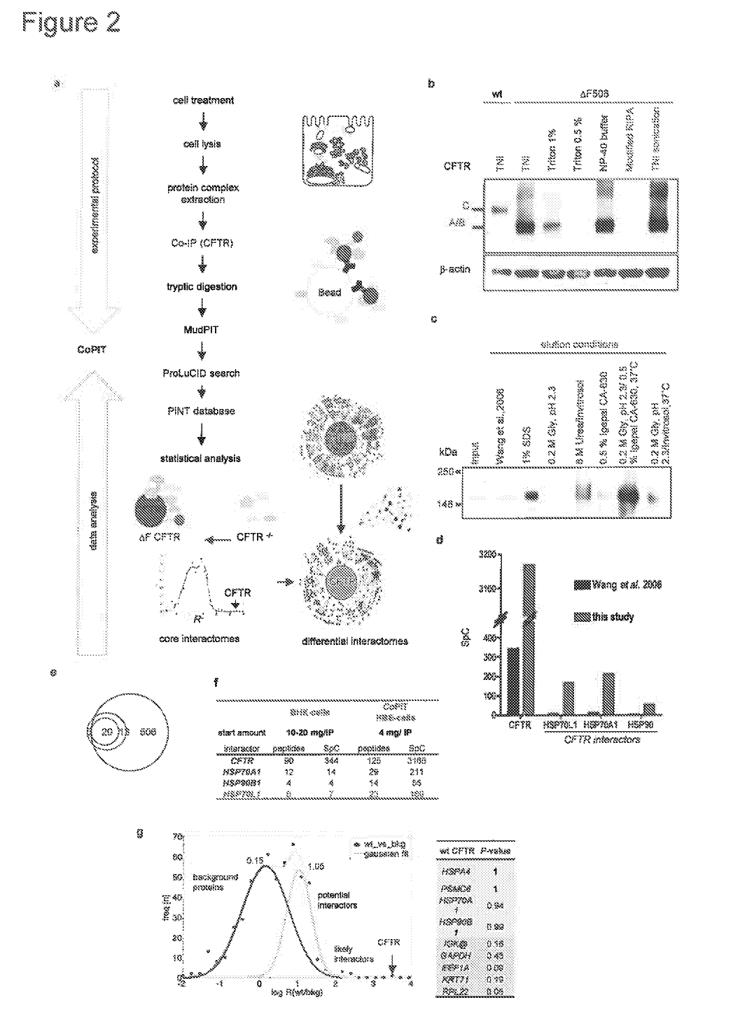

To identify interactions that potentially drive the disease phenotype, we developed Co Purifying Protein Identification Technology (CoPIT), an immuno-precipitation (IP) based proteomic-profiling approach of protein-protein interactions across different sample conditions. Using CoPIT, which increased CFTR yield by 30-100 fold, we first determined the changes that occur between the wt and ΔF508 CFTR interactome in isogenic HBF41o-(wt CFTR) and CFBE41o-(ΔF508 CFTR) bronchial epithelial cell lines derived from a CF patient (See, e.g., Bruscia et al., Gene Ther, 9, 683-685, 2002) (FIG. 2). Proteins mapping to 638 genes were classified as high-confidence interactors. ΔF508 CFTR and wt CFTR interactomes comprised 576 and 430 proteins, respectively, with an overlap of more than 85%. These 638 proteins form the core CFTR interactome, and represent direct as well as indirect CFTR. interactors. Additional 915 interactors with medium confidence scores and at least a ratio of 10:1 over background were further assembled into an extended interactome.

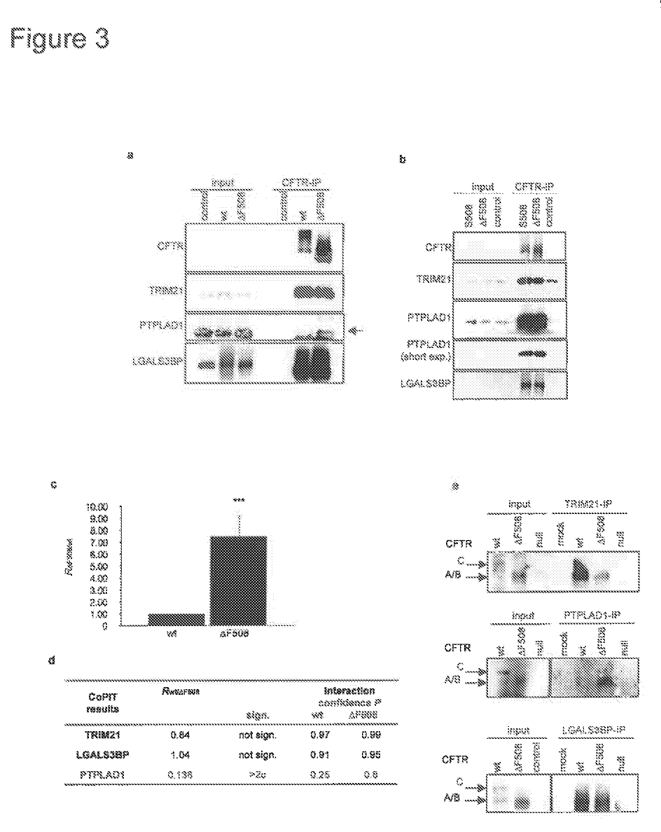

While the majority of proteins (368) in the core-interactome interact with both ΔF508 and wt CFTR, 209 differ significantly in the relative amounts recovered. Additional 208 and 62 interactors were detected only in ΔF508 CFTR and wt CFTR CoPIT experiments, respectively, and might represent interactors specific to or very highly enriched for either ΔF508 or wt CFTR. Protein expression profiling showed that the vast majority of observed differences between the ΔF508 and wt CFTR interactome are not due to altered expression levels of these proteins in the two cell lines. Thus, a ΔF508 CFTR mutation-specific interactome was identified, which is characterized mainly by gain of novel interaction partners. Alterations in protein networks revealed distinct differences in the biogenesis of wt and ΔF508 CFTR. In particular, we observed enhanced recruitment of specific chaperones like Hsp90 as well as enhanced protein degradation of ΔF508 CFTR mediated by a protein network, which differs vastly from the degradation and ER quality control network for wt CFTR and includes up to 25% of the ΔF508 CFTR specific interactions. While we recovered many of the proteins known to be involved in CFTR degradation, such as AMFR, STUB1 (CHIP) and VCP, we also identified several proteins that have been implicated previously in ERAD of other misfolded proteins but not of ΔF508 CFTR, including AUP1, SEl1L and FAF2. Several of these novel interactions, such as with the lectin-binding protein LGALS3BP and the E3-ligase TRIM21were confirmed by Co-IP followed by Western blot detection in bronchial epithelial cell lines and primary bronchial epithelial cells from CF patients (FIG. 3a-e). In addition, protein interactions implicated in translational control and mRNA decay, insertion of proteins into the ER (Translocation), N-glycosylation, protein transport and trafficking, anchoring at the plasma membrane, as well as endocytic recycling were strongly altered, suggesting that the entire CFTR biogenesis is affected by deletion of F508. An example of such re-routing is the association of ΔF508 CFTR with the ER quality control component and sugar transferase UGGT, leading to re-glucosylation of ΔF508 CFTR and eventual association with ERAD components, or the highly enhanced association of the co-chaperone PTPLAD1 with ΔF508 CFTR. Association of wt CFTR with components of Wnt and mTOR signaling pathways, and of ΔF508 CFTR with proteins involved in TGF-beta and JAK/STAT signaling suggests that cellular signaling is also affected by the F508 deletion. Taken together, these data suggest that the loss of ΔF508 CFTR function emerges from novel associations with multiple alternate protein complexes and cellular pathways that route ΔF508 CFTR differently than wt CFTR.

### Example 2

### Interactome Dynamics Upon Functional Rescue of ΔF508 CFTR at 30° C.

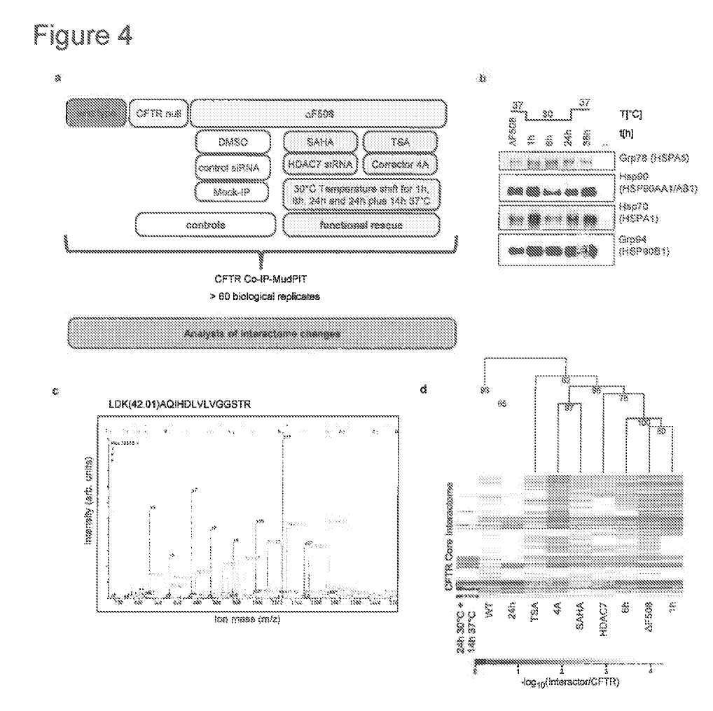

Culture at 26° C. to 30° C. promotes formation of the fully glycosylated form of ΔF508 CFTR (band C), incorporation into the plasma membrane and partial restoration of its channel activity. To probe the temporal dynamics of interactions with ΔF508 CFTR and identify the molecular mechanisms that facilitate full glycosylation and lead to functional rescue of ΔF508 CFTR at lower temperature, we monitored changes of the ΔF508 CFTR interactome at different time points during temperature shift to 30° C. (FIG. 4a). To this end, we first analyzed the ΔF508 CFTR-interactome by CoPIT after Short (1 h), intermediate (6 h), and long (24 h) incubation at 30° C., as well as upon reversal of the temperature shift (37° C. for 14 h after 24 at 30° C.). Changes in the interactome were tightly coupled to the appearance of fully glycosylated ΔF508 CFTR (Band C). While few interactome changes were observed after 1 h at 30° C., interactions with several proteins involved in ER quality control, like AIMP1 and AUP1, and in lysosomal targeting (LAMP1) were reduced, and a few new interactions were gained. Long term incubation at 30° C. abolished 186 of the 208 (89%) unique and highly confident interactions and the interactome was extensively remodeled with more than 65% of all interactions altered. The increased presence of band C was reflected in, first, reduced association of ΔF508 CFTR with degradation promoting proteins of ubiquitin-mediated pathways and ERAD, as well as of those involved in endocytic removal of plasma membrane proteins, second, by a more favorable folding environment marked by decreased recruitment of Hsp90 and glucose-regulated proteins, and third, by a markedly down-regulation of RNA processing (including mRNA decay) proteins like PABPC1 (33-fold).

Reversal of the temperature shift led to loss of fully glycosylated ΔF508 CFTR. However with only 20 interactions re-established, the ΔF508 CFTR interaction profile still clustered with that of wt CFTR. Interactions that mediate CFTR degradation from either the cell surface or ER, like E3-ubiquitin protein ligases AMFR (gp78) and STUB1, were re-gained first. Association of ΔF508 CFTR with RAB5B and RAB5A, which are involved in apical endocytosis and recycling, as well as with Erlin1 and Erlin2, which have been implicated in ERAD of IP3 receptors, was also restored. The experiment thus indicated that removal from the plasma membrane and subsequent degradation as well as degradation of newly synthesized ΔF508 CFTR in the ER is responsible for the rapid loss of fully glycosylated ΔF508 CFTR. Taken together, the temperature shift experiment revealed that the association of ΔF508 CFTR with the mutation-specific interactome and consequent alteration of CFTR biogenesis can be suppressed by temperature shift and thus may be responsible for the functional rescue.

### Example 3

### Interactome Remodeling Upon HDACi

It was reported that inhibition of HDAC activity leads to increased presence of fully glycosylated ΔF508 CFTR and partial functional rescue (Hutt et al., Nat. Chem. Biol. 6, 25-33, 2010). To identify the mechanisms by which HDAC inhibition mediates ΔF508 CFTR rescue, we monitored the interactome upon siRNA-mediated knockdown of HDAC7, or treatment with HDAC7 inhibitors. Specifically, CFBE41o-cells were treated with 100 nM Trichostatin A (TSA) or 5 μM Suberoylanilide hydroxamic acid (SAHA) for 24 h. Both of these HDAC inhibitors affect class I and II HDACs. In addition, HDAC7 was knocked down by siRNA mediated RNAi. All three treatments induced large-scale changes of the ΔF508 CFTR interactome, altering 35%-50% of all interactions. The treatment with 5 μM SAHA for 24 h induced the largest change to the ΔF508 CFTR interactome differentially down-replating 213 proteins by more than 3-fold (excluded by the circle in blue) and abolishing interactions with 81 proteins (not depicted in the network), while up-regulating interactions with 49 proteins by at least 3-fold (included within the red circle) and recruiting an additional 144 proteins to the ΔF508 CFTR interactome, of which 31 are also present in the wt CFTR interactome (innermost circle of proteins). In contrast, HDAC7 siRNA knockdown induced the least number of changes to the ΔF508 interactome, down-regulating 125 proteins by more than 3-fold (excluded by the circle in blue) and abolishing interactions with 108 proteins, while up-regulating interactions with only 13 proteins by more than 3-fold and recruiting an additional 48 proteins, of which 12 are present in the non-mutated CFTR interactome (included within the red circle). Those perturbations are effectively molecular pathways that permit a more wt CFTR-like biogenesis of ΔF508 CFTR.

The results also revealed that HDACi induced similar large-scale changes to the ΔF508 CFTR interactome as the temperature shift. More than 75% and almost 90% of interactions affected by TSA or HDAC7 knockdown were also altered by SARA treatment. In particular, HDACi abolished interactions that were either specific for or recruited preferentially to ΔF508 CFTR and restored a few wt CFTR-specific interactions, such as with the proteins NHERF1 and NEERF2, which can act as apical plasma membrane adapters for wt CFTR, and thus probably reflect enhanced ΔF508 CFTR stability at the plasma membrane.

Comparison of the interactions that were affected by temperature shift and HDACi identified trafficking, degradation and mRNA decay pathways required for ΔF508 CFTR rescue and pinpointed distinct differences in the mechanisms by which ΔF508 CFTR rescue is achieved. In contrast to temperature shift, TSA failed to reduce association with several protein disulfide isomerases that are involved in ER quality control. SARA treatment even enhanced association with ERAD component SEL1L, with E3-ligase SUGT1 and E3C ligase (UBE3C), which enhances proteasome processivity. We also identified additional lysosomal degradation proteins like Cathepsin B and TPP1 in the SARA interactome, probably reflecting failure of SAHA to fully prevent retrotranslocation and degradation of ΔF508 CFTR. Additionally, HDACi induced extensive changes to the ΔF508 CFTR associated cytoskeleton, which appear to have wide-ranging influence on anterograde and retrograde transport. Despite these changes to the interactome, the interaction profiles of ΔF508 CFTR upon treatment with HDACi or Cmpd 4a still clustered with the interaction profile of control ΔF508 CFTR rather than with wt CFTR (FIG. 4d). Further differences between temperature shift and HDACi mediated rescue included an inversely altered association of chaperone HSP70 and HSP90 family members with ΔF508 CFTR. While temperature shift only slightly affected association of ΔF508 CFTR with the HSP70 and Hsc70 chaperone machinery (1.35-fold less) it strongly reduced the association of ΔF508 CFTR with Hsp90 proteins (6.2-fold less). Conversely, HDACi strongly reduced the association of ΔF508 CFTR with detected Hsp70 family members (3.4-fold less) and affected association with Hsp90 proteins to a lesser degree (2.5-fold less). Reduced binding of chaperones to ΔF508 CFTR was independent of chaperone expression levels, which were either not influenced or up regulated by temperature shift or HDACi (FIG. 4b). However, enhanced acetylation of the ATPase domain of Hsp70 (HSPA1A) was observed upon HDACi, suggesting that the remodeling of the chaperone environment is induced by acetylation of Hsp70 (FIG. 4c). Acetylation of the ATPase domain may disrupt the Heat shock-Ubiquitin-Proteasome pathway, which controls mRNA decay. ΔF508 CFTR mRNA decay is possibly the pacemaker for the CF phenotype, as all treatments that induced ΔF508 CFTR rescue down regulate the association of a distinct set of more than 30 proteins that affect mRNA stabilization and decay, including PARPC1, YBX1, and UPF1.

Interestingly, a subset of seven ΔF508 CFTR specific interactions was neither corrected by temperature shift nor by SAHA. This subset includes members of the 26S proteasome (PSMC1, PSMD11), which induce protein aggregation and neurodegeneration if inhibited in their function, and PSMB8, a stress-inducible subunit of the 20S core proteasome, as well as the two co-chaperones BAG3 and DNAJB2. DNAJB2 inhibition leads to partial ΔF508 CFTR rescue. BAG3, whose binding to ΔF508 CFTR was significantly up-regulated immediately after temperature shift, mediates aggresome formation and selectively induces autophagy of misfolded proteins. Persistence of these interactions suggests that these proteins detect ΔF508 CFTR and channel it to autophagy and proteasomal degradation even under rescuing conditions. SURF4 has been implicated in vesicular trafficking and store-operated Ca²⁺ entry, whereas the molecular function of the last member of this subset, ERH, has remained enigmatic, but may be associated with RNA splicing.

### Example 4

### Interactor RNAi Restores ΔF508 Function

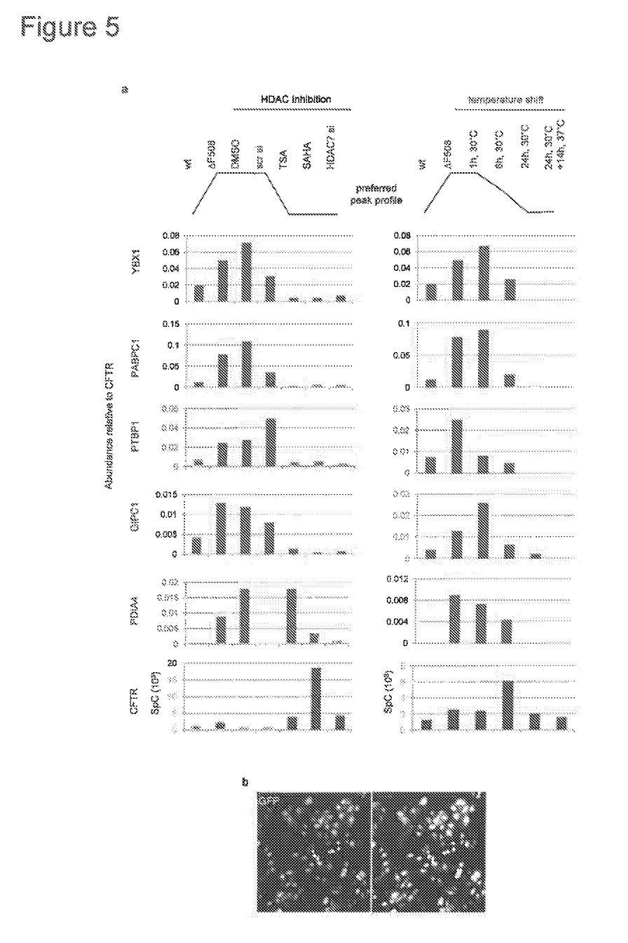

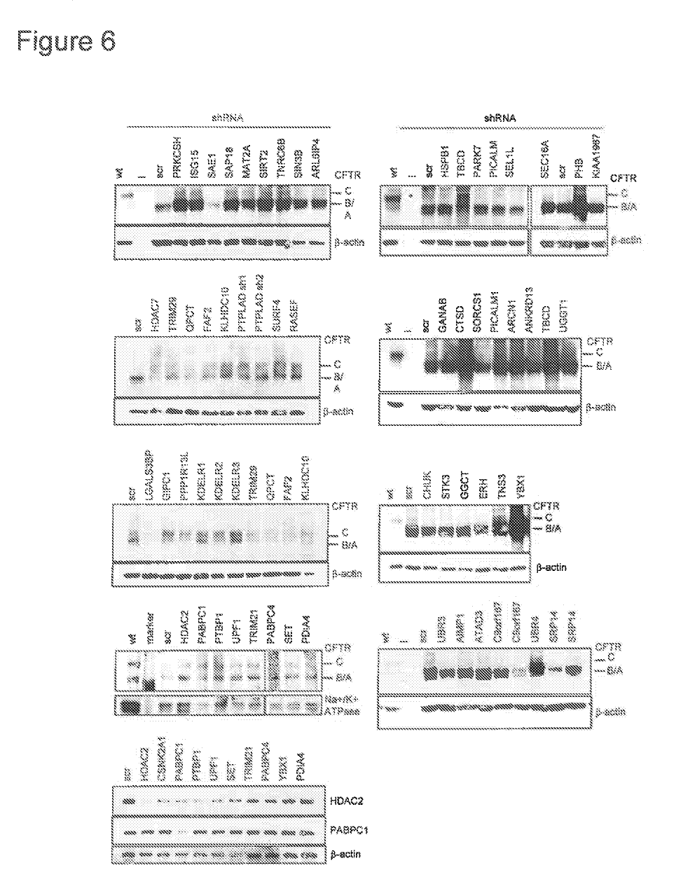

To assess the potential of rescuing the ΔF508 CFTR phenotype by blocking novel protein-protein interactions identified in this study, we performed an RNAi screen with validated shRNAs and monitored ΔF508 CFTR maturation and its glycosylation pattern by electrophoresis as a measure for ΔF508 MR rescue. A detailed description of the RNAi screen is provided in Example 5 below. In summary, a total of 52 proteins were tested including HDAC2 as positive and CSNK2A as negative controls (FIG. 5). Knockdown of 31 interactors promoted ΔF508 CFTR maturation, 6 proteins had minor to no effect, and knockdown of 17 proteins led to reduced ΔF508 CFTR stability and yield (FIG. 6). Many of the 31 novel interactors might sequentially control ΔF508 CFTR protein production and turn over as they belong to (1) a network associated with mRNA decay and co-translational control, (2) complexes affecting ΔF508 CFTR trafficking and endocytic recycling, (3) ER quality control and folding, or (4) the protein degradation network.

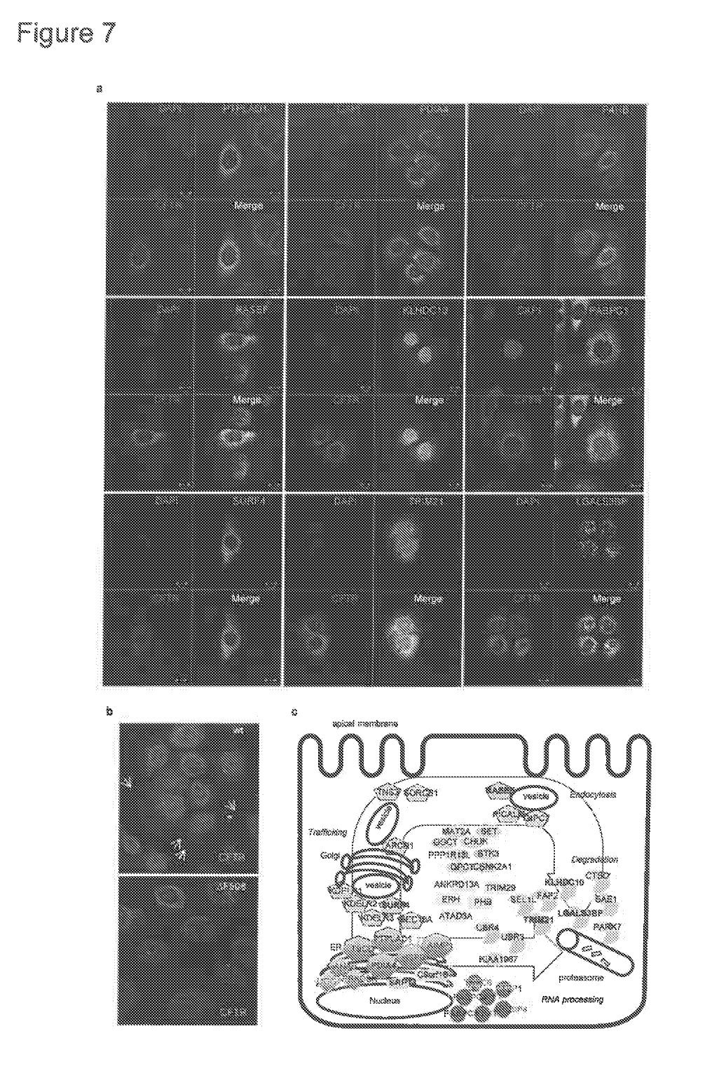

The subcellular interaction of ΔF508 CFTR with the top sub-networks or complexes was spatially resolved by co-immunostainings of nine binding partners that represent different cellular compartments according to Gene Ontology (FIG. 7a-c), Prolyl-4-hydroxylase (P4HB), an ER and plasma membrane marker, PDIA4, which recognizes unfolded protein regions, and PTPLAD1, which exhibits Hsp90 co-chaperone activity, co-localized with ΔF508 CFTR in the ER. Co-staining was also observed with SLAT 4, which is found in the early secretory pathway, ERGIC, and Golgi, as well as with the GTPase RASEF, which is potentially involved in membrane trafficking, Co-staining of ΔF508 CFTR with KLHDC10 and TRIM21, which are involved in degradation, and with PABPC1, which is involved in RNA processing, was observed in the nuclear periphery, LGALS3BP, which is part of the KLHDC10-FAF2 degradation complex and which negatively influenced ΔF508 CFTR stability, only partially co-localized with ΔF508 CFTR in vesicular structures.

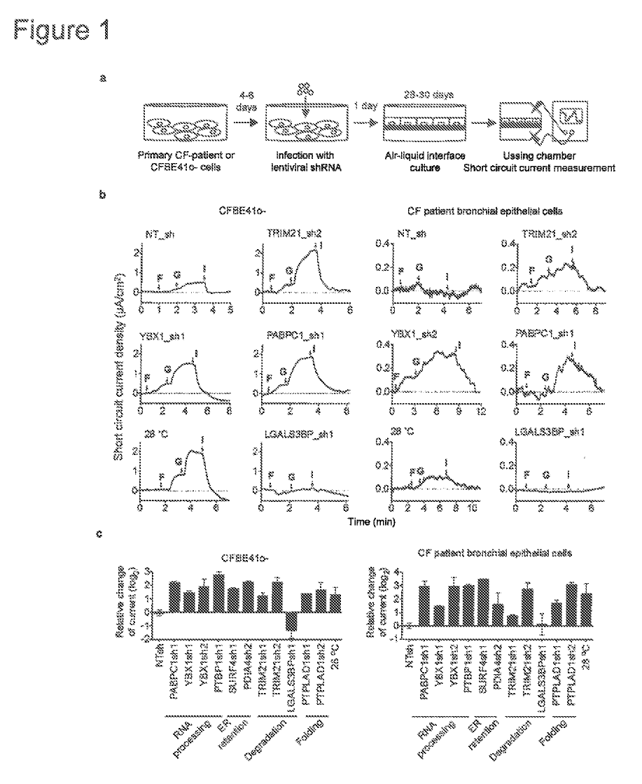

To further evaluate the therapeutic potential of interactors that influenced ΔF508 CFTR maturation in CFBE41o-cells in the RNAi screen, we assessed rescue of ΔF508 CFTR channel function for eight interactors that bind preferentially to ΔF508 CFTR and/or were dynamically regulated by temperature shift and HDACi. Each interactor represents either the RNA decay and co-translational control network (PABPC1, PTBP1, YBX1), the degradation network (LGALS3BP, TRIM21) or is a potential novel component of ER quality control (PDIA4, SURF4, PTPLAD1). Primary human bronchial epithelial cells from healthy donors or CF patients and CFBE41l -cells were differentiated into epithelial cultures at an air-liquid interface (ALI) and ΔF508 CFTR channel function was determined by electrophysiology in an Ussing chamber (FIG. 1a).

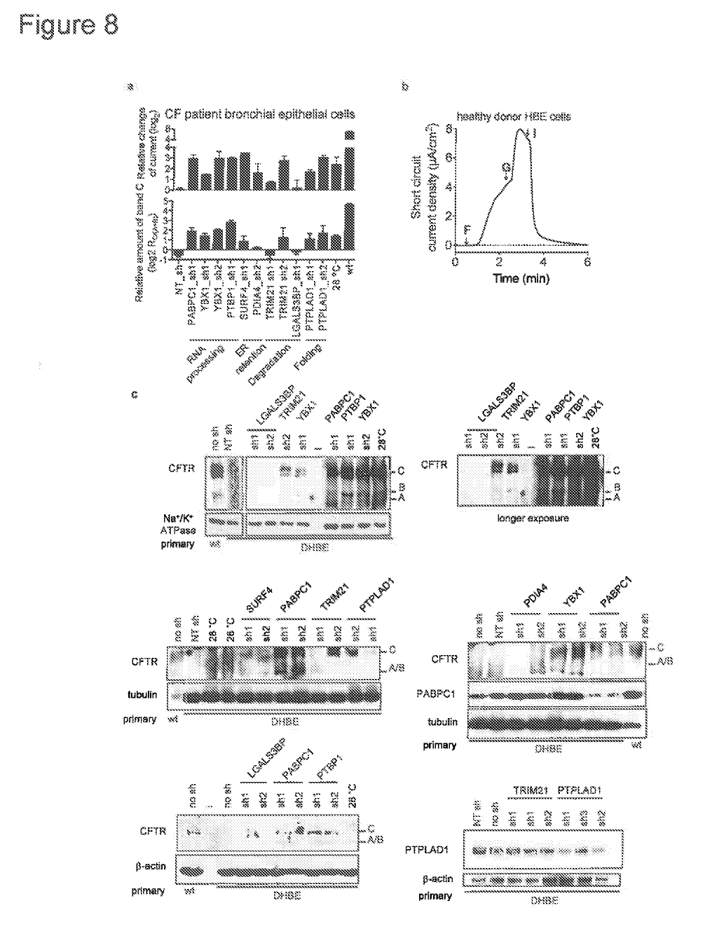

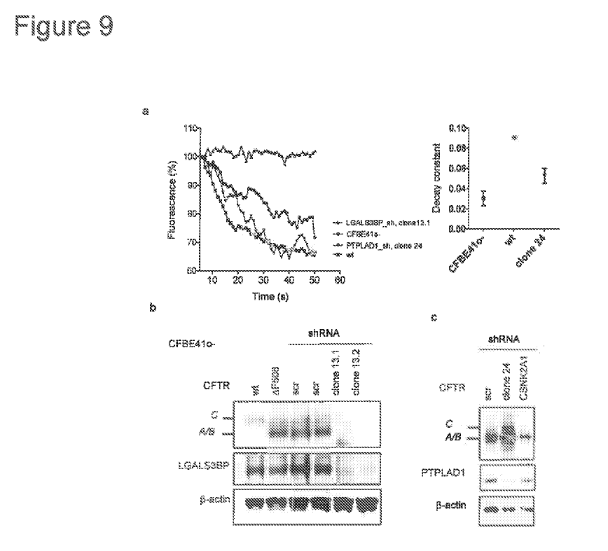

Knockdown of seven interactors enhanced forskolin/genistein-stimulated ΔF508 CFTR channel activity at the apical plasma membrane up to 8- to 12 fold over controls in primary CF epithelia and by about 4.5- to 7-fold in CFBE41o-epithelia, which is comparable to rescue by temperature shift (FIG. 1b-c). As determined by Western blot, knockdown of seven of the eight interactors also led to a clearly visible ΔF508 CFTR signal in the primary ALI cultures after differentiation for 28 d and induced band C formation similar to temperature shift, which correlates well with the increase in ΔF508 CFTR activity observed in the Ussing chamber measurements (FIG. 8). In the case of LGALS3BP knock down, no CFTR signal was detected in primary CF bronchial epithelial cells by Western blot and we failed to detect ΔF508 CFTR-specific chloride current in CFBE41o-epithelia or primary CF bronchial epithelia. Complete loss of ΔF508 CFTR in CFBE41o-cells that constitutively express an LGALS3BP shRNA (clone 13) showed that LGALS3BP is critical for ΔF508 CFTR stability. Furthermore, no CFTR chloride channel activity was measured upon LGALS3BP knockdown in a halide sensitive YFP assay, whereas upon stable knockdown of PTPLAD1 (clone 24), CFTR chloride channel function was greater than in parental CFBE41o-cells (FIG. 9). Our results show that reduction of protein levels of the other seven interactors rescues channel function of ΔF508 CFTR and thus we conclude that modulation of interactors can be a promising route to correction of the ΔF508 CFTR defect.

### Example 5

### Sub-Networks of Interactors Selected for RNAi

Candidates for the RNAI screen were selected according to the following criteria: (1) target interaction profiles (FIG. 5a), (2) abundance of candidate target proteins, (3) confidence of belonging to the core interactome, (4) significance of change upon temperature shift or HDACi (>=2σ cutoff), as well as (5) relational information about protein function and subcellular localization. Upon knockdown of the candidates, appearance of fully glycosylated ΔF508 CFTR (band C) was used to assess completion of glycosylation in the Golgi as an indicator for rescue of ΔF508 CFTR maturation, whereas the amount of total ΔF508 CFTR protein relative to control was used to assess changes in ΔF508 CFTR protein production. It can be argued that an increase in band B alone is likely to also lead to an increase in band C as a consequence of increased ΔF508 MR protein. However, increase in band B is not necessarily accompanied by the appearance of band C as shown by the example of ARL6IP4 and SIN3B knockdown. In addition, several target proteins showed increased ratios of band C to band A/B without or only marginal concomitant increase in band B.

Many of the 31 novel interactors whose knockdown promoted ΔF508 CFTR maturation belong to distinct protein complexes or form distinct sub-networks, which can be grouped into five categories:

(1) mRNA Decay and Co-Translational Control Network

The screen surprisingly identified a remarkable number of proteins involved in mRNA stabilization and translation that were overrepresented in ΔF508 CFTR IPs and whose interaction was strongly sensitive to both temperature shift and HDACi. Five of the 15 most consistently regulated and more abundant CFTR interactors in this group were poly-A binding protein, cytoplasmic 1 (PABPC1), poly-A binding protein, cytoplasmic 4 (PABPC4), polypyrimidine tract binding protein 1 (PTBP1), UPF1 regulator of nonsense transcripts homolog (yeast) (UPF1), and Y box binding protein 1 (YBX1), —none of which have been associated with CFTR biogenesis before. These five proteins form an extensive network together with other identified RNA processing proteins with similar interaction profiles as well as with proteins involved in translational control and the N-end-rule pathway. Strikingly, all of these proteins are implicated in translational silencing and can control mRNA degradation through divergent pathways of small RNA-mediated silencing, poly-A induced degradation or by stimulating exonuclease-mediated mRNA degradation. It is also known that PABP (PABPC1) is required for deadenylation. Since the knockdowns of PABPC1, PABPC4, PTBP1, UPF1 and YBX1 all strongly increased appearance of band C and some also increased band B, we suggest that this network controls ΔF508 CFTR translation by regulating its mRNA stability, perhaps depending on the progress of insertion into the ER membrane. Indeed, we identified three AU-rich elements in the 3′UTR and four AUUUA pentamers within its coding sequence (NM_000492, nt 372-376, nt 494-498, nt, 2884-2886, nt 4071-4075, nt 4806-4810, nt 5533-5537, nt 5698-5710, and (Baudouin-Legros et al., 2005) which are preserved in CFBE41o-cells. These elements are prime regulators of RNA stability and can repress translation. In addition, a translational inhibitor of labile inRNAs, TIA-1, which recognizes AU-rich elements and other mRNA destabilizing elements (polypyrimidine tracts) was detected only in the ΔF508 CFTR IPs.

### (2) Novel Hsp90 Chaperone Complex and ER Quality Control Components

The next step in CFTR biogenesis that was affected by shRNA knockdown of interactors, is the protein folding process and ER quality control. We uncovered that disturbing the interaction of ΔF508 CFTR with the protein tyrosine phosphatase-like A domain containing 1 (PrPLAD1) protein is critical for correct CFTR maturation, —most likely by influencing CFTR folding since PTPLAD1, also known as B-IND1, exhibits Hsp90 co-chaperone activity, is capable of binding to FKBP8 and is involved in recycling of the Hsp90 chaperone complex. Its knockdown strongly enhanced ΔF508 CFTR maturation, suggesting that it may limit the availability of the HSP90 chaperone complex for folding of ΔF508 CFTR or alternatively the accessibility of the folding machinery to ΔF508 CFTR. Another ΔF508 CFTR interactor which is potentially involved in modulating the ΔF508 CFTR folding process, is the protein disulfide isomerase family A member 4 (PDIA4), PDIA4 is part of an ER complex containing BiP, GRP94, UDP-GT, GRP170 and PDI that recognizes unfolded protein regions and subsequently retains such proteins in the ER, presumably for re-folding.

The enhanced maturation of ΔF508 CFTR upon knockdown of PDIA4 suggests that PDIA4 is probably involved in partial unfolding of the protein by reducing the disulfide bonds normally formed in the CFTR protein and is part of the ER-quality control that channels misfolded proteins to ERAD. In line with disturbed folding and enhanced ER quality control, we also detected enhanced association of the ER-resident enzymes UGGT1, PRKCSH, and GANAB, which are involved in N-glycosylation and influence interaction of glycoproteins with the lectin-chaperones calnexin and cafreticulin. We therefore tested knockdown of PRKCSH, GANAB and UGGT1. While knockdown of UGGT1 and GANAB did not significantly improve ΔF508 CFTR maturation, knockdown of PRKCSH, which encodes a subunit of glucosidase H, positively influenced ΔF508 CFTR maturation. However, its effect was rather weak when compared to rescue mediated by knockdown of other interactors, indicating that it is not enough to disturb ER quality control for correction of the CF defect.

(3) Sub-Complexes Influencing ΔP508 CFTR trafficking

The RNAi screen further identified several potential small sub-complexes that affect ΔF508 CFTR trafficking. One potential sub-complex is the Surf4-KDEL complex. The Surfeit 4 (SURF4) protein specifically and abundantly interacted with ΔF508 CFTR and its knockdown induced band C, the fully glycosylated and mature form of CFTR, without concomitant increase in band B. In S. cerevisiae the Surf4 homologue ERvp29 functions as a cargo receptor involved in protein sorting, and its knockout induces stabilization of misfolded soluble proteins. In mammalian cells, Surf4 co-localizes with KDEL receptors and is part of the early secretory pathway and its knockdown affects retrograde transport from the cis-Golgi to ERGIC. Since also the knockdown of KDELR1, KDELR2 and KDELR3 in CFBE41o-cells each stabilized ΔF508 CFTR and led to enhanced maturation of ΔF508 CFTR, Surf4 and KDEL-receptors might form a small network that retains ΔF508 CFTR and is involved in ΔF508 CFTR retrograde transport together with COP proteins. Additionally, knockdown of glutaminyl-peptide cyclotransferase (QPCT) influenced formation of band C. QPCT Isoenzymes have been identified in the Golgi and late secretory pathway and the observed effect on ΔF508 CFTR maturation may be due to altered retention of CFTR, or altered aggregation as inhibition of QPCT has been shown to reduce plaque formation and improve memory and learning in mouse models of Alzheimer's disease by preventing the formation of pyroglutamate residues, which block the N-terminus and enhance the aggregation propensity of beta-amyloid peptides. In addition, the RNAi screen identified two more interactors that potentially influence CFTR maturation by altering its trafficking, namely the PDZ domain containing protein GIPC1, and the RAS and EF-hand domain containing GTPase (RASEF). Knockdown of both GIPC1 and RASEF was found to enhance the ratio of band C to band A/B. GIPC1 has been described as endocytic adapter, which regulates trafficking of cell surface receptors and their lysosomal degradation. The function of RASEF has been little characterized, but it belongs to the Rab GTP Ase family, which contains important regulators of membrane trafficking and thus may influence ΔF508 CFTR trafficking.

### (4) ΔF508 CFTR Degradation Network

The Co-IP experiments also allowed us to define a novel, potential ΔF508 CFTR-specific degradation network. The ΔF508 CFTR specific interactome is highly enriched for proteins involved in ubiquitin mediated degradation and approximately 25% of the ΔF508 CFTR-specific interactions occur with proteins directly associated with the ubiquitin pathway or the ERAD pathway mirroring mis-processing and enhanced degradation of ΔF508 CFTR. It is well established that ΔF508 CFTR is readily degraded, but only part of the proteins are known that are involved in this process. The acquired data reveal this network for the first time and suggest that alternative pathways, in addition to the known components of the classical ERAD pathway, are involved both in wt and ΔF508 CFTR degradation and are potentially functionally redundant. A novel CFTR interactor, whose knockdown caused rescue of ΔF508 CFIR, was the potential E3-ligase TRIM21. TRIM21 is one of the most abundant interactors and binds equally to non-mutated and ΔF508 CFTR. Furthermore, knockdown of U-box domain 8 (FAF2, also called UBXD8), ketch domain containing 10 (KLHDC10) and ubiquitin protein ligase E3 component n-recognize 4 (UBR4) all protected CFTR from degradation, and permitted partial maturation as visualized by appearance of band C. KLHDC10 and FAF2 have recently been described as Cullin2 interactors, and may be associated with the E3-ubiquitin-ligase-complex Zyg11B-Cul2-TCEB1. In addition, FAF2 has been shown to bind lectin, galactoside-binding, soluble, 3 binding protein (LGALS3BP), which—if knocked down—decreased CFTR stability dramatically to the point where ΔF508 CFTR was no longer detectable by Western blot in CFBE41o-cells, suggesting that it counter-balances CFTR degradation by yet unknown mechanisms. In contrast, UBR4 is an E3-ligase that is part of the N-end ride pathway that recognizes destabilizing N-terminal residues according to the N-end rule and subsequently interacts with the ubiquitin system and Cul2 to deliver target proteins to the proteasomal degradation machinery. In addition, UBR4, also named p600, localizes to the ER in CNS neurons and has been identified as a calmodulin binding protein important for membrane morphogenesis. Thus, UBR4 might be important for coupling ΔF508 CFTR to the degradation machinery in the ER and the data suggest that degradation of ΔF508 CFTR might take place as early as its N-glycosylation occurs. Although none of these proteins have been described as interacting with non-mutated CFTR, or ΔF508 CFTR, FAF2 has been implicated in the dislocation of misfolded glycoproteins. Other E3-ligases and ubiquitin-associated proteins were also only detected in ΔF508 CFTR-IPs like Itch and Trim32 or UBXN1, UBAC2 and the E1-ligase UBA1, which catalyzes the first step of the ubiquitin conjugation process and recruits the E2.

### Example 6

### Small Molecule QPCT Inhibitors Rescue CFTR Channel Activity

In addition to RNAi mediated knockdown of the CFTR interactors, we also examined effects of some small molecule inhibitors of an interactor protein on rescue of ΔF508 CFTR cellular activities (e.g., channel function) in CFBE cells. In these studies, we employed two small molecule inhibitors against QPCT, N-ω-acetylhistamine (N-[2-(1H-Imidazol-4-yl)ethyl]acetamide); CAS: 673-49-4) and 1-benzylimidazole (1-Benzyl-1H-imidazole; CAS: 4238-71-5). Two derivative compounds of 1-benzylimidazole, 3-(3,4-dimethoxyphenyl)-1-(3-imidazol-1-ylpropyl)thiourea (PDB150; Pubchem CID 6539196) and 1-(3,4-Dimethoxyphenyl)-N-(3-(5-methyl-1H-imidazol-1-yl)propyl)cyclopropanecarbothioamide (Compound 11; Pubchem CID 44549233; CHEMBL583368), were also included in the studies.

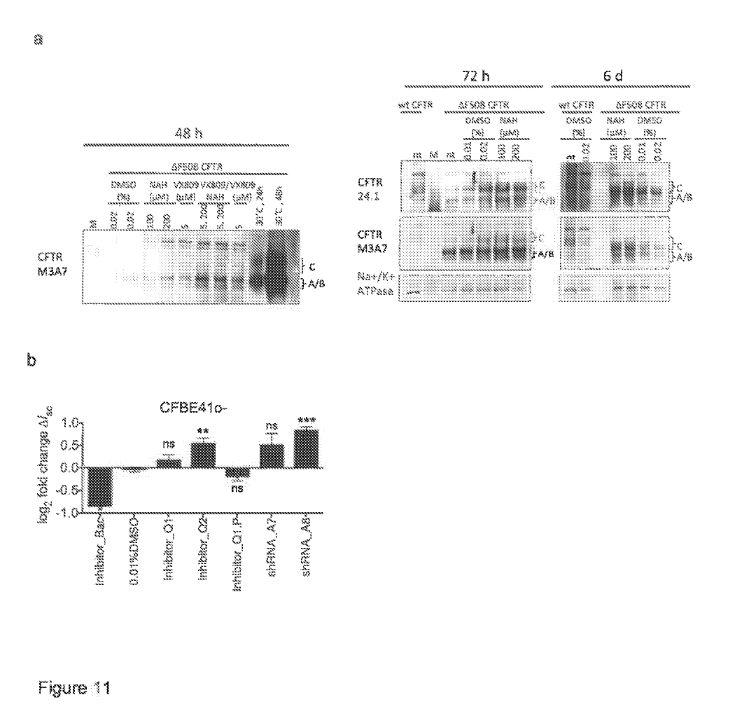

Results from these studies are shown in FIG. 11. Specifically, treatment for 24 h with ≥200 μM N-ω-acetylhistamine dissolved in DMSO increased production of fully glycosylated ΔF508 CFTR (Band C), and increased the ratio of Band C to Band B of ΔF508 CFTR in CFBE cells as determined by western blotting. The increase was larger after longer incubation times (48 h, 72 h) (See FIG. 11a). Also of note is that no cell toxicity was observed. In addition, treatment for 24 h with 500 μM N-ω-acetylhistamine dissolved in DMSO significantly increased ΔF508 MR channel activity by 1.5-fold over the DMSO control as measured by Ussing chamber measurement in CFBE cells. See FIG. 11b (“Inhibitor Q2”).

QPCT inhibitor 1-benzylimidazole was also examined for rescuing ΔF508 CFTR function. It was observed that treatment for 24 h with 100 μM 1-benzylimidazole dissolved in DMSO also increased ΔF508 am channel activity over the DIMSO control as measured by Ussing chamber measurement in CFBE cells. See FIG. 11b (“Inhibitor Q1”). Longer treatment time with this compound will likely further increase ΔF508 CFTR channel activity. We further examined the ability of the derivative compounds of I-benzylimidazole to restore ΔF508 CFTR cellular activities. For example, we observed that treatment for 24 h with 100 nM to 5 μM of Compound 11 dissolved in DMSO increased ratio of Band C to Band B of ΔF508 CFTR in UBE cells as determined by western blotting. Again, no cell toxicity was observed in the treatment.

### Example 7

### Materials and Methods

This example describes some of the material and methods that were employed in the exemplified embodiments herein.

Cell lines and cell culture: Human bronchial epithelial cells (CFBE41o-) carrying the ΔF508 CFTR mutation, or HBE41o-cells harboring a wt CFTR allele, and isogenic CFTR null cells (CFBE41o-null) were kindly provided by Dr. J. Clancy (University of Alabama, Birmingham, Ala.). Cells were cultured at 37° C., 5% CO₂ in Advanced-MEM (GIBCO, Carlsbad, Calif.) supplemented with 1% Penicillin/Streptomycin (GIBCO), 10% fetal bovine serum (GIBCO) and 2 mM L-Glutamine (GIBCO) and appropriate selective antibiotics. Cells were treated with 100 nM Trichostatin A (TSA, Sigma-Aldrich, St, Louis, Mo.), 5 μM Suberoylanilide hydroxamic acid (SAHA, Cayman Chemicals, Ann Arbor, Mich.), 15 μM N-[2-(5-Chloro-2-methoxyphenylamino)-4′-methyl-[4,5′]bithiazolyl-2′-yl]-benzamide (Cmpd 4a, C4, Cystic Fibrosis Foundation, www.cftrfolding.org/CFTReagents.htm) or vehicle (DMSO) for 20 h before immunoprecipitation. For siRNA-mediated knockdown of HDAC7, CFBE41o-cells were transfected with Lipofectamine RNAiMAX (Invitrogen, Carlsbad, Calif.) and 50 nM of validated HDAC7-specific sRNA (Ambion, Austin, Tex.) or scrambled control siRNA (Ambion) according to the manufacturers' protocol. The medium was changed the next day and cells harvested 72 h post-transfection. Primary bronchial epithelial cells were obtained from the Cystic Fibrosis Center at University of Alabama, Alabama according to IRB regulations and from Lonza (Walkersville, Md.), and were cultured in complete BEGM medium (Lonza) at 37° C., 5% CO₂ for up to three passages, starting with passage 0.

Lentiviral mediated knock down of target proteins: Lentiviral particles harboring shRNA sequences specific for the target proteins were generated in HEK293T cells using the Mission® shRNA system with validated shRNAs from commercial source (Sigma-Aldrich, St. Louis, Mo.) following standard protocols known in the art. See, e.g., Tiscornia et al., Nat. Protoc, 1, 241-245, 2006. CFBE41o-cells were infected with lentiviral particles for 16 h and cultivated for additional 48 h prior to harvest. Lentivirus production and infection is covered under TSR approval #01-13-10-07 and all steps were carried out in a BSL2/3 certified laboratory. Rescue of ΔF508 CFTR was monitored by Western blotting followed by immunodetection of CFTR using rat monoclonal 3G11 antibody. The TRC ID numbers for the shRNAs used for knockdown of some of the identified ΔF508 CFTR interactors, their specific target sequences, and the target interactor proteins are shown in Table 1.

TABLE 1
shRNA used for knockdown of ΔF508 CCFTR interactors

| | | Legacy Clone | | SEQ ID |
|---|---|---|---|---|
| Interactor | Clone ID | Name | Target Seeq | NO: |
| PDIA4 | TRCN0000049333 | NM_004911.3- | GCTTGTGTTGACCAAAGAGAA | 1 |
| | | 779s1c1 | | |
| PDIA4 | TRCN0000289674 | NM_004911.4- | CCTGAGAGAAGATTACAAATT | 2 |
| | | 1269s21c1 | | |
| SURF4 | TRCN0000153266 | NM_033161.2- | GCTCTTTGCCATCAACGTATA | 3 |
| | | 763s1cs | | |
| SURF4 | TRCN0000158134 | NM_033161.2- | CTTCGGGCTCTTTGGAATCAT | 4 |
| | | 412s1c1 | | |
| PTBP1 | TRCN0000231418 | NM_002819.3- | GCTTCTGCAGCAAACGGAAAT | 5 |
| | | 193S21C1 | | |
| PTBP1 | TRCN0000231420 | NM_002819.3- | GCGTGAAGATCCTGTTCAATA | 6 |
| | | 1262S21C1 | | |
| YBX1 | TRCN0000007948 | NM_004559.2- | GCTTACCATCTCTACCATCAT | 7 |
| | | 1095s1c1 | | |
| YBX1 | TRCN0000315307 | NM_004559.3- | CCAGTTCAAGGCAGTAAATAT | 8 |
| | | 565s21c1 | | |
| TRIM21 | TRCN0000010839 | NM_003141.x- | GAGTTGGCTGAGAAGTTGGAA | 9 |
| | | 555s1c1 | | |
| TRIM21 | TRCN0000003986 | NM_003141.x- | TGGCATGGTCTCCTTCTACAA | 10 |
| | | 1349s1ca | | |
| LGALSBP | TRCN0000029414 | NM_005567.2- | CGGAAGTCACAACTGGTCTAT | 11 |
| | | 1455s1c1 | | |
| LGALSBP | TRCN0000372837 | NM_005567.3- | GAGCGCTCAGCTTCAAGAAAT | 12 |
| | | 2068s2c1 | | |
| PABPC1 | TRCN0000074639 | NM_002568.3- | CCGCACCGTTCCACAGTATAA | 13 |
| | | 2019s1c1 | | |
| PABPC1 | TRCN0000293599 | NM_002568.3- | AGCTGTTCCCAACCCTGTAAT | 14 |
| | | 1674s21c1 | | |
| PPP1R13L | TRCN0000022209 | NM_006663.1- | CCAACTACTCTATCGTGGATT | 15 |
| | | 1524s1c1 | | |
| PPP1R13L | TRCN0000022212 | NM_006663.1- | CCCTACCCACAAGAAACAGTA | 16 |
| | | 1120s1c1 | | |
| QPCT | TRCN0000034592 | NM_012413.2- | GCCATTGCTGATAGAGCGATA | 17 |
| | | 224s1c1 | | |
| QPCT | TRCN0000034593 | NM_012413.2- | GCATCTGATACCGTCTCCTTT | 18 |
| | | 965s1c1 | | |
| PTPLAD1 | TRCN0000072998 | NM_016395.1- | CCCAGTAACATTCCTGAATTT | 19 |
| | | 1369s1c1 | | |
| PTPLAD1 | TRCN0000073000 | NM_016395.1- | GCTTCGTTACACTCTGTGGAT | 20 |
| | | 977s1c1 | | |

Western Blotting and Immunofluorescence: Protein lysates were prepared as described for CoPIT denatured in SDS sample buffer for 15 min at 37° C. for detecting CFTR or for 5 min at 95° C., separated by SDS-PAGE and transferred onto nitrocellulose (Protran, Schleicher&Schuell, Germany). The following primary antibodies were used: Rat monoclonal antibody against CFTR (3G11), mouse monoclonal antibodies against CFTR (24.1, ATCC; M3A7, Chemicon, Temecula, Calif.) and Vactin (AC-15, Sigma), rabbit polyclonal antibodies against HDAC2 (9928S, Cell Signaling, Danvers, Mass.), PABPC1 (ab21060, Abeam, Cambridge, Mass.), anti-galectin-3BP (AF 2226, R&D Systems, Minneapolis, Minn.), anti-PTPLAD1 (WH0051495M1, Sigma), anti-52 kDA RoISSA (sc-25351, Santa Cruz, Calif.) and anti-Na⁺/K⁺ ATPase a Antibody (H-300, sc28800, Santa Cruz). Horseradish-peroxidase conjugated secondary antibodies (Jackson ImmunoResearch, West Grove, Pa.) were detected with enhanced chemiluminescence reagent (ECL, Pierce, Rockford, Ill.). For Immunofluorescence, CFBE41o-cells fixed with 4% Paraformaldehyde were permeabilized with 0.1% Triton X100, blocked in 10% FBS in 1× PBS for 1 h at room temperature and incubated with the following antibodies for 4 h at RT: anti-CFTR (3G11), anti-galectin-3BP (R&D Systems, AF2226), anti-PTPLAD1 (Sigma, WH0051495M1), anti-KLHDC10 (Sigma, HPA020119), anti-52 kDa Ro/SSA (Santa Cruz, sc-25351), anti-Rab45 (Santa Cruz, se-81925), anti-Surfeit4 (Santa Cruz, sc-107304), anti-Erp72 (Abeam, ab82587, Enzo ADI-SPS-720), anti-PABPC1 (Abeam, ab21060) and anti-P4HB (3501S, Cell Signaling). AlexaFluor 488-, DyLight 488-, or DyLight 549-conjugated secondary antibodies (Jackson ImmunoResearch) were used for detection of the primary antibodies. Nuclei were counterstained with DAPI (Molecular Probes, Invitrogen). Photographs of cells mounted in ProLong Gold antifade reagent (Molecular Probes, linvitrogen) were taken with a laser scanning confocal microscope LSM 710 (Zeiss) or Radiance 2100 Rainbow (Zeiss).

Premo™ Halide sensor assay: Chloride channel activity of CFTR was determined with a Premo™ Halide sensor assay (Invitrogen) measuring quenching of a halide-sensitive yellow-fluorescent protein (YFP) variant (Venus YFP). To this end, HBE41o-, CFBE41o-, and CHBE41o-cell lines stably transduced with shRNA lentivirus were infected with the Bacman gene delivery system to introduce the YFP expression construct according to manufacturer's recommendations (Invitrogen). Subsequently, cells were seeded in glass bottom 96 or 24 well plates and cultivated overnight. Quenching of fluorescence by Iodide influx was measured on single cell level with a Radiance 2100 Rainbow laser scanning confocal microscope (Zeiss) according to the protocol initially established by Galietta et al. ⁷². Briefly, before time-lapse recording cells were pre-incubated with 50 μM Genistein. Cells with sufficient YFP fluorescence were then selected and data acquisition was started with a frame speed of 0.5 s to 1.0 s. After 5 s, Nal was added to 0.1 M final concentration and chloride channel activity was further stimulated by addition of Forskolin (20 μM). Acquired data were analyzed with Matlab (www.mathworks.com) and Prism (GraphPad Software, Inc.), and decay curves were fitted over the time course. At least 10 individual cells for each cell line were recorded per experiment.

Ussing chamber ineasureinents: Primary human CF and control (wt) bronchial epithelial cells infected with Mission shRNA lentiviral particles with a multiplicity of infection (MOI) between 3 and 5 were plated on 12 mm Snapwell membranes (Corning, Cambridge, Mass.) coated with rat tail collagen I (BD Biosciences) at a density of 1×10⁵ cells/cm² and cultured in BEGM. Upon confluence, cells were maintained in B-ALI differentiation medium (Lonza) under air-liquid interface conditions for at least 21 d. Transepithelial resistance (TEER, R_{T}) of the ALT cultures was between 200 and 2700Ω/cm², and was measured with a Millicell ERS2 Volt-Ohmmeter (Millipore, Billerica, Mass.). Polarized cultures were mounted in EasyMount Ussing chambers (Physiological Instruments, San Diego, Calif.) and bathed bilaterally with Krebs-bicarbonate Ringer solution (140 mM Na⁺, 119.8 mM Cl⁻, 25 mM HCO₃⁻, 2.8 mM K⁺, 2.4 mM HPO₄, 0.4 mM PO₄, 1.2 mM Mg⁺, 1.2 mM Ca⁺, 5 mM Glucose) and the solution saturated with 95% O₂, 5% CO₂. The epithelial sodium channel was blocked with 100 μM Amiloride (Sigma-Aldrich). CFTR was stimulated by addition of Forskolin (10 μM) and Genistein (50 μM) to the apical side of the chamber followed by CFTR Inhibitor 172 (20 μM, EMD Biosciences, apical) to isolate the CFTR-specific, apical Cl⁻ current. Measurements were carried out at 37° C. and the short circuit current (I_{sc}) was recorded and analyzed with Acquire and Analyze 2.0 (Physiological Instruments).

CoPIT Co-Immunoprecipitation and sample preparation for LC/LC-MS/MS: Rat monoclonal anti-CFTR antibody (3G11) was coupled to ProteinG Sepharose 4 Fast Flow beads (GE Healthcare, Piscataway, N.J.) at 6 mg/ml packed beads and covalently crosslinked to the beads with 20 mM Dimethylpimelimidate (DMP, Pierce). CFBE41o- or HBE41o-cells from passage 5 to 19 were grown to confluence in Advanced-MEM supplemented with 10% FCS, 1% Penicillin/Streptomycin, 2 mM L-glutamine and appropriate antibiotics. Approximately 4×10⁷ or 1×10⁸ cells were harvested per IP, rinsed with PBS, lysed, CFTR protein complexes immuno-precipitated and prepared for mass spectrometric analysis according to the CoPIT protocol. Briefly, cells were lysed in 0.5% Igepal CA-630 (Sigma-Aldrich), 50 mM Trig pH 7.5, 250 mM NaCl, 1 mM EDTA and 1× Complete EDTA-free Protease Inhibitor mix (Roche, Switzerland) on ice. Following water-bath sonication insoluble material was removed by centrifugation (30 min, 14,000 rpm, 4° C.) and the supernatant pre-cleared by incubation with CIAB-Sepharose (GE Healthcare). The pre-cleared lysate was then incubated overni t at 4° C. with 50 μl (approx. 250 μg) of anti-CFTR 3G11 antibody covalently coupled to ProteinG-Sepharose. Immunoprecipitates were recovered by centrifugation (500×g, 5 min, 4° C.), washed three times with lysis buffer and two times with lysis buffer containing no detergent. Bound proteins were eluted twice with 0.2 M Glycine pH 2.3 and 0.5% Igepal CA-630 at 37° C. and precipitated (Eluate Methanol Chloroform, 1:4:1, v:v:v). The precipitate was washed with 95% Methanol and re-solobilized in 100 mM Tris pH 8.5 and 0.2% Rapigest (Waters, Milford, Mass.). Samples were reduced with 5 mM TCEP (Pierce), alkylated with 10 mM iodoacetamide (Pierce) and proteins digested overnight with 3 μg of sequencing-grade recombinant Trypsin (Promega, Madison, Wis.), Formic acid (9% final, v:v) was added to inactivate Rapigest (2 h at 37° C.), any precipitate removed by centrifugation (15 min, 14,000 rpm at RT), and samples reduced to near dryness in vacuo. To identify non-specific contaminating proteins, control IPs were carried out from (a) isogenic CFTR null cells to identify background that is recognized non-specifically by the 3G11 antibody and (b) by using mock-IPs, in which no antibody is coupled to the beads, to control for bead specific and cell specific background.

Expression profiling: Protein lysates from CFBE41o- and HBE41o-cells at the same passage number were prepared in TNI lysis buffer, precipitated (lysate: Methanol: Chloroform (1:4:1, v:v:v) and 100 μg of protein reduced, alkylated and digested with Trypsin as described above. Resulting peptides were labeled with 6-plex Tandem Mass Tag labeling reagent (Thermo-Fisher, San Jose, Calif.) according to manufacturers' recommendations. Subsequently, Rapigest was inactivated by acidification with 10% formic acid, insoluble precipitate removed by centrifugation (15 min, 14,000 rpm), and samples reduced to near dryness in vacuo.

LC/LC-MS/MS: Samples were analyzed by nano-ESI-LC/LC-MS/MS on an LTQ-Orbitrap XL. LTQ or Orbitrap Elite (Thermo Fisher, San Jose, Calif.) by placing the triphasic MudPIT column inline with an Agilent 1100 quaternary HPLC pump (Agilent, Palo Alto, Calif.) and separating the peptides in multiple dimensions with a modified 6-step gradient containing 0%, 20%, 40%, 60%, and 100% of Buffer C (500 mM ammonium acetate/5% acetonitrile/0.1% formic acid) over 12 h or a 10-step gradient (0%, 10%, 20%, 30%, 40%, 50%, 70%, 50%, 90%, 100% Buffer C) over 20 h as described previously ⁷³. Each full scan mass spectrum (400-2000 m/z) was followed by 6 (LTQ, LTQ-Orbitrap XL,) or 20 (Orbitrap Elite) data-dependent MS/MS scans at 35% normalized collisional energy and an ion count threshold of 1000 (LTQ-Orbitrap XL, Orbitrap Elite) or 500 counts (LTQ). Dynamic exclusion was used with an exclusion list of 500, repeat time of 60 s and asymmetric exclusion window of −0.51 and +1.5 Da. To avoid cross contaminations between the different samples, each sample was loaded onto a fresh column.

CoPIT data anal Raw-files were extracted with RawExtract (fields.scripps,edu/researchtools.php) and MS/MS spectra searched with ProLuCID (Xu et al., Mol. Cell. Proteomics 5, S174, 2006) against the human International Protein Index (IPI) database version 3.23, using a target-decoy approach in which each protein sequence is reversed and concatenated to the normal database (Elias et al., Nat. Methods 2, 667.-675, 2005). Search parameters were set to no enzyme specificity, 50 ppm precursor mass tolerance, and carboxyamidomethylation (m=57.021464 Da) as a static modification. Search results were filtered with DTA Select version 2.1 allowing for tryptic peptides only and a peptide false discovery rate (FDR) of less than 0.5%, usually corresponding to a protein false discovery rate of less than 1.0%. To uniformly control the FDR across samples in CoPIT, and eliminate potential comparison problems arising from the use of isoform-specific identifiers, sqt-files of replicate samples were filtered in one single DTASelect run and split again in corresponding replicate subsets for further analysis. Samples with non-sufficient recovery of the bait (<35 SpC) were excluded from further analysis. To remove redundancy, which is problematic for statistical analysis, IPI numbers were first converted to Entrez Gene Symbol using the X-REF Converter developed by RIKEN (http://refdic.rcai.riken.jp/tool/xrefconv.cgi) and manual annotation based on the Ensernhl release 43 (www.regulatorygenomics.org) and the highest PSM (peptide-spectrum match) value of all protein variants per gene and experiment retained. CoPIT assumes that proteins binding nonspecifically and non-selectively to carrier or antibody are detected with equal likelihood in experimental conditions (e) and control (c) as shown in Li et al., Analytical chemistry 75, 6648-6657, 2003; and Ranish et al., Nature genetics 36, 707-713, 2004. Ratios for proteins p were calculated as

$${r}_{pec} = {log}_{10} ( \frac{{\sum }_{i = 1}^{n} PSM \text{?}}{{\sum }_{i = 1}^{n} PSM \text{?}} ) , n$$

$$\text{?} \text{indicates text missing or illegible when filed}$$

equals number of experiments. Data were then plotted in Matlab, and a bimodal model to analyze the frequency distribution of all ratios r_{p ec} applied and fitted with a Gaussian of two terms

$$v \text{?} = {A}_{bg} e \text{?} + {A}_{sp} e \text{?}$$

$$\text{?} \text{indicates text missing or illegible when filed}$$

with a goodness of fit between 0.90≤R²≤0.98, where (bg) is background and (sp) bait specific interactors. Confidence values are calculated for each protein according to

$$P = erf [ \vert {r}_{pec} - {μ}_{bg} \vert / \sqrt{2 * ( {σ}_{{r}_{pec}}^{2} + {σ}_{bg}^{2} + σ \text{?} )} ]$$

$$\text{?} \text{indicates text missing or illegible when filed}$$

derived from the respective terms of the Gaussian fit. Proteins that were identified only in background control samples were eliminated from the analysis as obvious background contaminants. For a protein to be considered a potentially true interactor, we required further that it be detected in at least two independent biological replicates of the same condition to minimize random sampling errors and IDs. Fold change of a proteinp between two different experimental conditions was calculated according to

$${r}_{p} = {log}_{10} ( \text{?} ) . \\ \text{?} \text{indicates text missing or illegible when filed}$$

Errors for relative changes were calculated based on random error of measurement

$${σ}_{{r}_{p}} = \frac{1}{2} ( {log}_{10} ( 1 + \sqrt{\text{?}} ) + \vert {log}_{10} ( 1 - \sqrt{\text{?}} ) \vert )$$

$$\text{?} \text{indicates text missing or illegible when filed}$$

in CoPIT and if not indicated otherwise, the following significance definitions were used throughout all figures: (*) indicates (σᵣ_{ₚ}+log₁₆1.32)≤rₚ<(2σᵣ_{ₚ}+log₁₀1.32) and (**) indicates rᵣₚ≥(2σᵣ_{ₚ}+log₁₀1.32), wherein rₚ is the average relative ratio of the protein and σᵣ_{ₚ} is the random error of measurement.

Annotation data were derived from Uniprot Knowledge Base, Entrez Gene information, GO Miner and literature review on PubMed. Interactions between the identified interactors were obtained with the GeneMANIA 2.2 Piugin in Cytoscape 2.8.2 using physical interactions reported in BIOGRID-small scale studies, BIOGRID and BIND as well as Pathway information reported in Pathway Commons. Proteins, their connections and according functional annotation were then graphed in the Radial Topology Viewer 0.6, which was based on Medusa, whereby length of individual edges reflects a quantitative relationship with the bait such as enrichment over background.

Analysis of additional small networks was carried out using Osprey 1.2.0 and Ingenuity Pathway Analysis (IPA). Analysis of the expression profiling experiments was carried out in Census and the Integrated Proteomics pipeline (Integrated Proteomies Applications, Inc) using the TMT-option with 10 mDa tolerance and a minimum intensity threshold of 100,000 relative ion counts (Park et al., Nat Methods 5, 319-322, 2008). Statistical significance was determined with an unpaired t-test for differential expression (two-tailed and two-sample t-test or every protein). The volcano plot was generated with the biostatistics package in Matlab (Mathworks). The dataset was uploaded to Proteomics JNTegrator (PINT, unpublished, S.M.B.) for online accession by the scientific community at http://sealion.scripps.edulpint?project=CFTR (“CFTR” dataset). It includes all qualitative and quantitative data over all experimental conditions and replicates measured. In addition, PINT provides an advanced query and annotation system, including the retrieval of Uniprot annotations assigned to the proteins in the dataset.

CFTR Gore interactome hierarchical clustering analysis: The CFTR interaction profile of a given condition was represented by log₁₀ transformed ratios of core interactome protein abundances (sum of spectral counts across the replicates of that condition) and the abundance value of CFTR in that same condition. Hierarchical clustering of the different conditions was produced using the average linkage algorithm. The distance between two conditions was set to one minus their Pearson correlation. Heatmap representation was produced using gplots version 2.14.1 package and bootstrap values were obtained using the R package pvclust 1,2-2.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

1. A method for restoring proper folding and function of Cystic Fibrosis Transmembrane Conductance Regulator (CFTR) in a cell with phenylalanine 508 in-flame-deletion CFTR mutant (ΔF⁵⁰³ CFTR), comprising contacting the cell harboring said mutant CFTR with an agent that specifically downregulates expression level, inhibits cellular activity, or interrupts protein-protein interaction of a CFTR interactor protein, thereby restoring proper folding and function of CFTR in the cell; wherein the CFTR interactor protein is selected from the group consisting of glutaminyl-peptide cyclotransferase (QPCT), polyadenylate-binding protein 1(PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), E3 ubiquitin-protein ligase TRIM21 (TRIM21), protein-tyrosine phosphatase-like A domain-containing protein 1 (PTPLAD1), protein-glutamine gamma-glutamyltransferase 2 (TGM2), and RelA-associated inhibitor (PPP1R13).

2. The method of claim 1, wherein the agent specifically inhibits the cellular activity of the CFTR interactor protein.

3. The method of claim 2, wherein the CFTR interactor protein is an enzyme, and the agent specifically inhibits the enzymatic activity of the CFTR interactor protein.

4. The method of claim 2, wherein the CFTR interactor protein is glutaminyl-peptide cyclotransferase (QPCT), and the agent is a small molecule inhibitor of QPCT. The method of claim 4, wherein the agent is N-ω-acetylhistamine (systematic name: N-[2-(1H-Imidazol-4-yl)ethyl]acetamide, CAS: 673-49-4), 1-benzylimidazole (systematic name: 1-Benzyl-1H-imidazole; CAS: 4238-71-5), 3-(3,4-dimethoxyphenyl)-1-(3-imidazol-1-ylpropyl)thiourea, 1-(3,4-Dimethoxyphenyl)-N-(3-(5-methyl-1H-imidazol-1-yl)propyl)cyclopropanecarbothioamide, a derivative or analog compound thereof, or a pharmaceutically acceptable salt thereof.

6. The method of claim 5, wherein the derivative or analog compound has one or more mono- or multi valent groups substituted with a different mono- or multi valent group independently selected from the group consisting of: H; halogen; straight, cyclic or branched chain alkyl; straight, cyclic or branched chain alkenyl; straight, cyclic or branched chain alkynyl; halo-alkyl -alkenyl or -alkynyl; CN; CF₃; aryl and substituted aryl groups in which any or all H groups of the aryl ring is substituted with a different group; heterocyclic and substituted heterocyclic groups in which any or all groups of the aryl ring is substituted with a different group; carboxyl; carbonyl, alkoxyl; alkyloxyalkanes; alkoxycarbonyi; aryloxyl, heterocyclyloxyl; hydroxyl; amine; amide; amino; quaternary amino; nitro; sulfonyl; alkylamine; silyl, siloxyl; saturated C—C bonds; unsaturated C—C bonds; ester, ether, amino; amide, urethane, carbonyl, acetyl and ketyl groups; hetero atoms N, S and O; polymer groups; and amino acids.

7. The method of claim 5, wherein the derivative or analog compound has one or more hydrogens substituted with a lower alkyl group.

8. The method of claim 1, wherein the agent specifically downregulates expression level of the CFTR interactor protein.

9. The method of claim 8, wherein the agent is an inhibitory polynucleotide that suppresses expression of the CFTR interactor protein.

10. The method of claim 9, wherein the inhibitory polynucleotide is a short hairpin RNA (shRNA), a short interfering RNAs (siRiNA), a microRNAs (miRNA), or an anti-sense nucleic acid.

11. The method of claim 1, wherein the cell is a pulmonary epithelial cell.

12. The method of claim 1, wherein the cell is present in a subject.

13. A method for treating or ameliorating the symptoms of cystic fibrosis in a subject, comprising administering to the subject a pharmaceutical composition that comprises a therapeutic effective amount of an agent that specifically downrcgulates expression level or inhibits cellular activity of a CFTR interactor protein, thereby treating or ameliorating the symptoms of cystic fibrosis in a subject; wherein the CFTR interactor protein is selected from the group consisting of glutarninyl-peptide cyclotransferase (QPCT), polyadenylate-binding protein 1 (PABPC1), nuclease-sensitive element-binding protein 1 (YBX1), polypyrimidine tract-binding protein 1 (PTBP1), surfeit locus protein 4 (SURF4), protein disulfide isomerase A4 (PDIA4), E3 ubiquitin-protein ligase TRIM21 (TR1M21), protein-tyiosirre phosphatase-like A domain-containing protein 1 (PTPLAD1), protein-glutamine gamma-glutamyltransferase 2 (TGM2), and RelA-associated inhibitor (PPP1R13).

14. The method of claim 13, wherein the subject harbors Cystic Fibrosis Transmembrane Conductance Regulator (CFTR) mutant with phenylalanine 508 in-flame-deletion (ΔF⁵⁰⁸ CFTR).

15. The method of claim 13, wherein the agent specifically inhibits the cellular activity of the CFTR interactor protein.

16. The method of claim 15, wherein the CFTR interactor protein is an enzyme, and the agent specifically inhibits the enzymatic activity of the CFTR interactor protein.

17. The method of claim 15, wherein the CFTR interactor protein is glutaminyl-peptide cyclotransferase (QPCT), and the agent is a small molecule inhibitor of QPCT.

18. The method of claim 17, wherein the agent is N-ω-acetylhistamine (systematic name: N-[2-(1H-imidazol-4-yl)ethyl]acetamide, CAS: 673-49-4), 1-benzylimidazole (systematic name: 1-Benzyl-1H-1H-imidazole; CAS: 4238-71-5), 3-(3,4-dimethoxyphenyl)-1-(3 -imidazol-1-ylpropyl)thiourea, 1-(3,4 -Dimetboxyphenyl)-N-(3 -(5-methyl-1H-imidazol-1-yl)propyl)cyclopropanecarbothioamide, a derivative or analog compound thereof, or a pharmaceutically acceptable salt thereof.

19. The method of claim 13, wherein the agent specifically downregulates the expression level of the CFTR interactor protein.

20. The method of claim 19, wherein the agent is an inhibitory polynucleotide that suppresses expression of the CFTR interactor protein.

21. The method of claim 20, wherein the inhibitory polynucleotide is a short hairpin RNA (shRNA), a short interfering RNAs (siRNA), a microRNAs (miRNA), or an anti-sense nucleic acid.

22. A method for identifying an agent that is capable of restoring proper folding and function of ΔF⁵⁰⁸ CFTR, comprising (a) synthesizing one or more structural analogs of a lead compound selected from the group consisting of N-ω-acetylhistamine and 1-benzylimidazole, and (b) performing a functional assay on the analogs to identify an analog that has an improved biological or pharmaceutical property relative to that of the lead compound; thereby identifying an agent that is capable of restoring proper folding and function of ΔF⁵⁰⁸ CFTR.

23. The method of claim 22, wherein the improved biological or pharmaceutical property is an enhanced activity in inhibiting QPCT enzymatic activity.
